(19) United States
(12) Patent Application Publication
(10) Pub. No.: US 2025/0066733 A1
(43) Pub. Date: Feb. 27, 2025

(54) CARDIOVASCULAR CELL CO-CULTURE MEDIUM AND METHOD OF GROWING MULTIPLE CARDIOVASCULAR CELL TYPES

(71) Applicant: CEDARS-SINAI MEDICAL CENTER, Los Angeles, CA (US)

(72) Inventors: Arun SHARMA, Los Angeles, CA (US); Clive N. SVENDSEN, Pacific Palisades, CA (US)

(73) Assignee: CEDARS-SINAI MEDICAL CENTER, Los Angeles, CA (US)

(21) Appl. No.: 18/726,179

(22) PCT Filed: Jan. 5, 2023
(86) PCT No.: PCT/US2023/060137

§ 371 Date: Jul. 2, 2024

Related U.S. Application Data
(60) Provisional application No. 63/297,053, filed on Jan. 6, 2022.

(51) Int. Cl. C12N 5/077

(52) U.S. Cl. CPC C12N 5/0657; C12N 2500/38; C12N 2501/105; C12N 2501/11; C12N 2501/115; C12N 2501/165; C12N 2501/39; C12N 2501/91; C12N 2501/998; C12N 2506/45

(57) ABSTRACT

Described herein is a unique cell culture media that allows optimal growth of multiple cell types in the same system such as an organ-on-chip or organoid. Also described herein is use of the cell culture media in growing multiple cardiovascular cell types simultaneously in a single device or producing induced pluripotent stem cell (iPSC)-derived cardiac organoids containing iPSC derived-endothelial cells (iPSC-ECs) and iPSC derived-cardiomyocytes (iPSC-CMs). In some embodiments, a formulation for the cell culture media includes a base medium including glucose, a pH indicator, salts, amino acids, and vitamins; recombinant human albumin; L-ascorbic acid 2-phosphate; and at least one or more of insulin, vascular endothelial growth factor (VEGF), epidermal growth factor (EGF), basic fibroblast growth factor (bFGF), a synthetic analogue of insulin-like growth factor (IGF)-I, heparin, or hydrocortisone.

### CROSS-REFERENCE TO RELATED APPLICATIONS

This application includes a claim of priority under 35 U.S.C. § 119(e) to U.S. provisional patent application No. 63/297,053, filed Jan. 6, 2022, the entirety of which is hereby incorporated by reference.

### STATEMENT REGARDING FEDERALLY SPONSORED RESEARCH OR DEVELOPMENT

This invention was made with government support under Grant No. NS105703 awarded by National Institutes of Health. The Government has certain rights in the invention.

### FIELD OF INVENTION

This invention relates to cell culture media and methods of using the media for culturing various types of cardiovascular cells.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Sometimes, a single culture medium that is optimal for cultivation of various types of cells is required when the various types of cells are grown simultaneously in the same system. For example, a device or system, such as a microfluidic organ-on-a-chip system, for culturing two different types of cells need to have a single common culture medium that is optimal for cultivation of both types of cells. In one example, a device having a plurality of channels may include one type of cells in one channel and another type of cells in another channel, and only a single common culture medium can be provided to both types of cells.

The cells of the cardiovascular system include endothelial cells, vascular smooth muscle cells, lymphatic endothelial cells, cardiomyocytes, and atherosclerosis cells. Each of these cells may require different types of culture media for optimal growth and maintenance.

Among the various cardiovascular cells, primary human cardiomyocytes (CMs) could be used for preclinical evaluation of drug cardiotoxicity, but their access is limited and their long-term maintenance is a technical challenge. Also, since adult human CMs are mitotically inactive, they cannot be expanded in vitro to obtain enough cells for drug screening. The most predictive models for drug cardiotoxicity need to reproduce the complex spatial distribution of the CMs, endothelial cells (ECs), and support cells of the adult human heart. However, primary human heart preps are too costly, too difficult to maintain, and too low-throughput to be implemented early during drug development.

Moreover, studies of cardiomyocyte culture require a high quality cardiomyocyte population, successful cell isolation and maintenance during culture remain challenging. Specific features of the culture methods are needed to ensure long term survival and in vivo properties of the cultured cells. The main considerations for choice of medium are buffering, ionic composition and nutritional supplements. Myocyte growth medium should contain all the components necessary for the optimal growth of human CMs. For example, various cardiomyocyte growth media, such as RPMI 1640+B27 with insulin, are commercially available.

Endothelial Cell Growth Medium 2 (EGM2), which is commercially available, is a low-serum (2% V/V) medium that lacks Endothelial Cell Growth Supplement (ECGS, bovine hypothalamic extract) but contains Insulin-like Growth Factor (Long R3 IGF) and Vascular Endothelial Growth Factor (VEGF). Generally, VEGF leads to higher endothelial cell proliferation in culture. EGM2 is optimized for the cultivation of endothelial cells from large blood vessels and for primary human cells.

To maintain the integrity and function of different types of cells, for example, endothelial cells and cardiomyocytes, that are grown together simultaneously, an all-in-one cardiovascular cell co-culture medium is necessary. Such a medium must provide nutrients that are required for growth of both cardiomyocytes and endothelial cells, including critical proteins and growth factors. Otherwise, separate media must be provided independently to maintain the integrity and functionality of endothelial cells and cardiomyocytes. The separate media have unique growth factors that are optimized for either cardiomyocytes or endothelial cells, and thus, one of these media cannot satisfy nutrient requirements for both endothelial cells and cardiomyocytes that are grown together simultaneously. One possible solution has been to mix these separate media types into a 50/50 medium that includes both. However, such a mixed medium may not be optimal for both cell types. Therefore, there is a great need in the art for a cell culture medium that allows for optimal growth of multiple cell types in the same system. Further, there is a need for an all-in-one culture medium that is chemically defined and made of known components, unlike other approaches that use serum or other undefined substances.

### SUMMARY OF THE INVENTION

The present disclosure provides a formulation including: a base medium comprising glucose, a pH indicator, salts, amino acids, and vitamins; recombinant human albumin; L-ascorbic acid 2-phosphate; and at least one or more of insulin, vascular endothelial growth factor (VEGF), epidermal growth factor (EGF), basic fibroblast growth factor (bFGF), a synthetic analogue of insulin-like growth factor (IGF)-I, heparin, or hydrocortisone.

According to some embodiments, the base medium is Roswell Park Memorial Institute (RPMI) 1640 medium with L-glutamine.

According to some embodiments, a concentration of glucose in the base medium is about 2,000 mg/L. According to some embodiments, the pH indicator includes phenol red and a concentration of phenol red in the base medium is about 5 mg/L. According to some embodiments, the salts are inorganic salts including calcium nitrate (Ca(NO₃)₂ 4H₂O), magnesium sulfate (MgSO₄) (anhyd.), potassium chloride (KCl), sodium bicarbonate (NaHCO₃), sodium chloride (NaCl), and sodium phosphate dibasic (Na₂HPO₄) anhydrous. According to some embodiments, concentrations of calcium nitrate (Ca(NO₃)₂ 4H₂O), magnesium sulfate (MgSO₄) (anhyd.), potassium chloride (KCl), sodium bicarbonate (NaHCO₃), sodium chloride (NaCl), and sodium phosphate dibasic (Na₂HPO₄) anhydrous in the base medium are about 100 mg/L, about 48.84 mg/L, about 400 mg/L, about 2,000 mg/L, about 6,000 mg/L, and about 800 mg/L, respectively.

According to some embodiments, the amino acids include reduced glutathione. According to some embodiments, the amino acids further include Glycine, L-Arginine, L-Asparagine, L-Aspartic acid, L-Cystine, L-Glutamic Acid, L-glutamine, L-Histidine, L-Hydroxyproline, L-Isoleucine, L-Leucine, L-Lysine hydrochloride, L-Methionine, L-Phenylalanine, L-Proline, L-Serine, L-Threonine, L-Tryptophan, L-Tyrosine disodium salt dehydrate, and L-Valine. According to some embodiments, concentrations of each amino acid is in a range of about 1-about 500 mg/L, a concentration of L-glutamine being the highest and a concentration of L-Tryptophan being the lowest among the concentrations of the amino acids.

According to some embodiments, the vitamins includel biotin, choline chloride, D-calcium pantothenate, folic acid, niacinamide, para-aminobenzoic acid, pyridoxine hydrochloride, riboflavin, thiamine hydrochloride, vitamin B12, and i-inositol. According to some embodiments, concentrations of each vitamin is in a range of about 0.001-about 175 mg/L, a concentration of i-inositol being the highest and a concentration of vitamin B12 being the lowest among the concentrations of the vitamins.

According to some embodiments, amounts of the recombinant human albumin and L-ascorbic acid 2-phosphate in the formulation are in ranges of about 100 μg-about 2500 μg/mL and about 45 μg-about 1,100 μg, respectively, per 500 mL of the base medium. According to some embodiments, the amounts of recombinant human albumin and L-ascorbic acid 2-phosphate in the formulation are in ranges of about 400 μg-about 600 μg/mL and about 150 μg-about 300 μg/mL, respectively, per 500 mL of the base medium.

According to some embodiments, amounts of insulin, heparin, and hydrocortisone in the formulation are in ranges of about 1 μg-about 25 μg/mL, about 5 μg-about 110 μg/mL, and about 0.05 μg-about 1 μg/mL, respectively, per 500 mL of the base medium. According to some embodiments, amounts of insulin, heparin, and hydrocortisone in the formulation are in ranges of about 3 μg-about 7 μg/mL, about 15 μg-about 30 μg/mL, and about 0.1 μg-about 0.5 μg/mL, respectively, per 500 mL of the base medium.

According to some embodiments, the synthetic analogue of IGF-I is LONG® R3-IGF-I, and an amount of LONG® R3-IGF-I in the formulation is in a range of about 4 ng-100 ng/mL or about 10 ng-30 ng/ml per 500 mL of the base medium.

According to some embodiments, amounts of VEGF, EGF, and bFGF in the formulation are in ranges of about 0.1 ng-about 2.5 ng/ml, about 1 ng-about 25 ng/ml, and about 2 ng-50 ng/ml, respectively, per 500 mL of the base medium. According to some embodiments, amounts of VEGF, EGF, and bFGF in the formulation are in ranges of about 0.3 ng-about 0.7 ng/ml, about 3 ng-about 7 ng/mL, and about 5 ng-15 ng/mL, respectively, per 500 mL of the base medium.

According to some embodiments, the formulation is cell culture media optimized for human induced pluripotent stem cell (hiPSC)-derived cardiovascular cell co-culture systems including 2D co-culture, cardiac organoids, and cardiac organ chips.

The present disclosure provides a method of growing multiple cardiovascular cell types in a device comprising multiple channels, wherein different types of cells are in different channels. In various embodiments, the method includes circulating the above-described formulation through the multiple channels such that the different types of cells are grown in the formulation simultaneously without requiring different culture media in each channel having a different cell type.

According to some embodiments, induced pluripotent stem cell-derived endothelial cells (iPSC-ECs) and induced pluripotent stem cell-derived cardiac cells (iPSC-CCs) are in a first channel and a second channel among the multiple channels, or in the second channel and the first channel, respectively; the iPSC-ECs and the iPSC-CCs are in different channels; and the formulation is optimized for growth of both the iPSC-ECs and the iPSC-CCs.

According to some embodiments, the iPSC-CCs are induced pluripotent stem cell-derived cardiomyocytes (iPSC-CMs); induced pluripotent stem cell-derived cardiac fibroblasts (iPSC-CFs) are in the first or second channel among the multiple channels or the iPSC-ECs, the iPSC-CMs, and iPSC-CFs are in different channels; and the formulation is optimized for growth of the iPSC-ECs, the iPSC-CMs, and the iPSC-CFs.

According to some embodiments, the iPSC-ECs are induced pluripotent stem cell derived-vascular endothelial cells (iPSC-vECs); and the iPSC-CCs are induced pluripotent stem cell derived-cardiomyocytes (iPSC-CMs), induced pluripotent stem cell-derived fibroblast cells (iPSC-FCs), or induced pluripotent stem cell-derived smooth muscle cells (iPSC-SMCs).

According to some embodiments, the iPSC-ECs are induced pluripotent stem cell-derived vascular endothelial cells (iPSC-vECs); and the iPSC-CCs are induced pluripotent stem cell-derived cardiomyocytes (iPSC-CMs).

For example, the device is a microfluidic organ-on-chip. For example, the iPSC-ECs are human iPSC-ECs (hiPSC-ECs). For example, the iPSC-CFs are human iPSC-CFs (hiPSC-CFs).

According to some embodiments, the first and second channels include polydimethylciloxane, or the different channels comprise polydimethylciloxane. For example, the first channel and the second channel are microfluidic channels, or the different channels are microfluidic channels.

The present disclosure provides a method of producing induced pluripotent stem cell (iPSC)-derived cardiac organoids containing iPSC-derived endothelial cells (iPSC-ECs) and iPSC-derived cardiac cells (iPSC-CCs). In various embodiments, the method includes harvesting iPSC-ECs and iPSC-CCs in a well plate under a sterile cell culture environment; resuspending the harvested iPSC-ECs and iPSC-CCs in the above-described formulation to generate cardiac organoids; centrifuging the well plate to aggregate the organoids at a bottom of each well of the well plate; and culturing the organoids with the formulation, changing the formulation three times a week. The formulation is optimized for growth of both the iPSC-ECs and the iPSC-CCs.

According to some embodiments, the iPSC-ECs are induced pluripotent stem cell-derived vascular endothelial cells (iPSC-vECs); and the iPSC-CCs are induced pluripotent stem cell-derived cardiomyocytes (iPSC-CMs), induced pluripotent stem cell-derived fibroblast cells (iPSC-FCs), or induced pluripotent stem cell-derived smooth muscle cells (iPSC-SMCs).

For example, the iPSC-ECs are induced pluripotent stem cell-derived vascular endothelial cells (iPSC-vECs); and the iPSC-CCs are induced pluripotent stem cell-derived cardiomyocytes (iPSC-CMs). For example, the iPSC-ECs are human iPSC-ECs (hiPSC-ECs). For example, the iPSC-CCs are human iPSC-CCs (hiPSC-CCs).

According to some embodiments, the harvesting iPSC-ECs and iPSC-CCs or iPSC-CMs includes harvesting iPSC-ECs and iPSC-CCs or iPSC-CMs in a ratio of 1:9. According to some embodiments, the harvesting iPSC-ECs and iPSC-CCs or iPSC-CMs includes harvesting about 2,000 or 2,000 iPSC-ECs and about 18,000 or 18,000 iPSC-CCs or iPSC-CMs per organoid.

According to some embodiments, the harvested iPSC-ECs and iPSC-CCs or iPSC-CMs are resuspended in about 200 μL or 200 μL of the formulation in each well of the well plate such that cardiac organoids with about 20,000 or 20,000 cells are generated. According to some embodiments, each generated organoid has about 2,000 or 2,000 iPSC-ECs and about 18,000 or 18,000 iPSC-CCs or iPSC-CMs. According to some embodiments, the harvesting iPSC-ECs and iPSC-CCs or iPSC-CMs includes harvesting about 1,000 or 1,000 iPSC-ECs and about 9,000 or 9,000 iPSC-CCs or iPSC-CMs per organoid.

According to some embodiments, the harvested iPSC-ECs and iPSC-CCs or iPSC-CMs are resuspended in about 200 μL or 200 μL of the formulation in each well of the well plate such that cardiac organoids with about 10,000 or 10,000 cells are generated. According to some embodiments, each generated organoid has about 1,000 or 1,000 iPSC-ECs and about 9,000 or 9,000 iPSC-CCs or iPSC-CMs.

According to some embodiments, the well plate is centrifuged for about 10 or 10 seconds at about 200× or 200× gravity to aggregate the organoids at the bottom of the wells. According to some embodiments, the aggregated organoids are cultured in the formulation at 37° C. under 5% CO₂ condition indefinitely. According to some embodiments, the changing the formulation three times a week includes conducting a 75% media change of the formulation to prevent aspiration of the organoids from wells.

The present disclosure provides a method of producing induced pluripotent stem cell (iPSC)-derived cardiac organoids containing iPSC-derived endothelial cells (iPSC-ECs), iPSC-derived cardiac fibroblasts (iPSC-CFs), and iPSC-derived cardiomyocytes (iPSC-CMs). In various embodiments, the method includes: harvesting iPSC-ECs, iPSC-CFs, and iPSC-CMs in a well plate under a sterile cell culture environment; resuspending the harvested iPSC-ECs, iPSC-CFs, and iPSC-CMs in the formulation of any one of claims 1-19 to generate cardiac organoids;

centrifuging the well plate to aggregate the organoids at a bottom of each well of the well plate; and culturing the organoids with the formulation, changing the formulation three times a week. The formulation is optimized for growth of the iPSC-ECs, the iPSC-CFs, and the iPSC-CMs.

For example, the iPSC-ECs are induced pluripotent stem cell-derived vascular endothelial cells (iPSC-VECs). For example, the iPSC-ECs are human iPSC-ECs (hiPSC-ECs), the iPSC-CFs are human iPSC-CFs (hiPSC-CFs), and the iPSC-CMs are human iPSC-CMs (hiPSC-CMs). For example, the iPSC-ECs are human iPSC-ECs (hiPSC-ECs), the iPSC-CFs are human iPSC-CFs (hiPSC-CFs), and the iPSC-CMs are human iPSC-CMs (hiPSC-CMs).

According to some embodiments, the harvesting iPSC-ECs, the iPSC-CFs, and iPSC-CMs includes harvesting iPSC-ECs, the iPSC-CFs, and iPSC-CMs in a ratio of 1:1:8. According to some embodiments, the harvesting iPSC-ECs, the iPSC-CFs, and iPSC-CMs comprises harvesting about 2,000 or 2,000 iPSC-ECs, harvesting about 2,000 or 2,000 iPSC-CFs, and about 16,000 or 16,000 iPSC-CMs per organoid.

According to some embodiments, the harvested iPSC-ECs, iPSC-CFs, and iPSC-CMs are resuspended in about 200 μL or 200 μL of the formulation in each well of the well plate such that cardiac organoids with about 20,000 or 20,000 cells are generated. According to some embodiments, each generated organoid has about 2,000 or 2,000 iPSC-ECs, about 2,000 or 2,000 iPSC-CFs, and about 16,000 or 16,000 iPSC-CMs. According to some embodiments, the harvesting iPSC-ECs, iPSC-CFs, and iPSC-CMs includes harvesting about 1,000 or 1,000 iPSC-ECs, about 1,000 or 1,000 iPSC-CFs and about 8,000 or 8,000 iPSC-CMs per organoid. According to some embodiments, the harvested iPSC-ECs, iPSC-CFs, and iPSC-CMs are resuspended in about 200 μL or 200 μL of the formulation in each well of the well plate such that cardiac organoids with about 10,000 or 10,000 cells are generated.

According to some embodiments, each generated organoid has about 1,000 or 1,000iPSC-ECs, about 1,000 or 1,000 iPSC-CFs, and about 8,000 or 8,000 iPSC-CMs. According to some embodiments, the well plate is centrifuged for about 10 or 10 seconds at about 200× or 200× gravity to aggregate the organoids at the bottom of the wells. According to some embodiments, the aggregated organoids are cultured in the formulation at 37° C. under 5% CO₂ condition indefinitely. According to some embodiments, the changing the formulation three times a week includes conducting a 75% media change of the formulation to prevent aspiration of the organoids from wells.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts cardiac organoids grown in a medium in accordance with various embodiments of the present invention.

FIG. 2 shows a graph showing beat rates for cardiac organoids grown in the Heart Media for 15 weeks in accordance with various embodiments of the present invention.

FIG. 3 shows a graph showing beat rates of 2D hiPSC-CMS plated in the Heart Media for 9 weeks in accordance with various embodiments of the present invention.

FIG. 4 shows a graph comparing functionality data among hiPSC-cardiomyocytes/endothelial cell co-cultured with three different types of media including a medium in accordance with various embodiments of the present invention.

FIG. 5 shows a graph comparing viability data among hiPSC-cardiomyocytes/endothelial cell co-cultured with three different types of media including a medium in accordance with various embodiments of the present invention.

FIG. 6 shows a graph showing average beat rates of CM/EC/CF organoid seeds.

FIG. 7 and FIG. 8 show images of CM/EC/CF organoids.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

As used herein the term “organ chip” (also referred to as “organ-on-chip”) refers to a microfluidic culture device are capable of recapitulating the microarchitecture and functions of living organs.

Human induced pluripotent stem cell-derived cardiac spheroids (hiPSC-cardiac spheroids) represent a powerful three-dimensional (3D) model for examining cardiac physiology and for drug toxicity screening. Recent advances in self-organizing, multicellular cardiac organoids highlight the capability of directed stem cell differentiation approaches to recapitulate the composition of the human heart in vitro. Using hiPSC-derived cardiomyocytes (hiPSC-CMs), hiPSC-derived endothelial cells (hiPSC-ECs), and hiPSC-derived cardiac fibroblasts (hiPSC-CFs) is advantageous for enabling tri-cellular crosstalk within a multi-lineage system and for generating patient-specific models. Culture media consisting of factors needed to simultaneously maintain hiPSC-CMs, hiPSC-ECs, and hiPSC-CFs is used to produce the spheroid system.

Described herein is a culture medium optimized for co-culturing multiple cardiovascular cell types, for example, human induced pluripotent stem cell-derived heart muscle cells, or cardiomyocytes (hiPSC-CMs), endothelial blood vessel cells (hiPSC-ECs), and cardiac fibroblasts (hiPSC-CFs) among others, although the cell types are not limited to hiPSC-CMs, hiPSC-ECs, and hiPSC-CFs. In particular, culture media disclosed herein can be used in a human organ-chip for modeling disease pathology, including infection disease, examining drug toxicity and efficacy, and aids the development of new therapeutics and assist clinicians in choosing treatment regimens.

Also described herein are various protocols to illustrate the methods for conducting small molecule-mediated differentiations of hiPSCs into cardiomyocytes, endothelial cells, and cardiac fibroblasts, as well as how to assemble the fully-integrated cardiac spheroids.

Further described herein is a serum-free chemically defined cell culture medium composition/formulation. Thus, all components of the formulation are known and the formulation could be adapted for good manufacturing practice (GMP) purposes. In particular, the formulation is optimized for simultaneous growth of various cardiovascular cell types, the formulation containing all the essential nutrients required by the various types of cells. For example, the formulation can be used in a combination of induced pluripotent stem cell-derived cardiomyocytes (iPSC-CMs) and/or induced pluripotent stem cell-derived endothelial cells (iPSC-ECs) and/or induced pluripotent stem cell-derived cardiac fibroblasts (iPSC-CFs) (particularly, hiPSC-CMs, hiPSC-vascular ECs (hiPSC-VECs), and hiPSC-CFs) which can serve as an in vitro platform for assessing disease pathology, including infectious disease, evaluate drug efficacy, toxicity, cardiotoxicity and cardioprotection.

Also described herein are a method of growing multiple cardiovascular cell types and a method of producing induced pluripotent stem cell (iPSC)-derived cardiac organoids containing iPSC-ECs, iPSC-CMs, and/or iPSC-CFs, using the formulation. The ability to grow various cardiovascular cell types simultaneously using the single culture media will potentially reduce costs and burdens that may be required for using independent culture media separately for different cell types or mixing different types of culture media.

### Formulations

Various embodiments of the present invention provide for a formulation including a base medium comprising glucose, a pH indicator, salts, amino acids, and vitamins; recombinant human albumin; L-ascorbic acid 2-phosphate; and at least one or more of insulin, vascular endothelial growth factor (VEGF), epidermal growth factor (EGF), basic fibroblast growth factor (bFGF), a synthetic analogue of insulin-like growth factor (IGF)-I, heparin, or hydrocortisone.

In various embodiments, the base medium is Roswell Park Memorial Institute (RPMI) 1640 medium with L-glutamine, for example, Gibco/ThermoFisher Catalog number 11875085.

In various embodiments, a concentration of glucose in the base medium is about 500 mg/L, about 1,000 mg/L, about 1,500 mg/L, about 2,000 mg/L, about 2,500 mg/L, about 3,000 mg/L, about 3,500 mg/L, or about 4,000 mg/L.

In various embodiments, a concentration of glucose in the base medium is about 2,000 mg/L.

In various embodiments, the pH indicator is phenol red and a concentration of phenol red in the base medium is about 1 mg/L, about 2 mg/L, about 3 mg/L, about 4 mg/L, about 5 mg/L, about 6 mg/L, about 7 mg/L, about 8 mg/L, about 9 mg/L, or about 10 mg/L.

In various embodiments, the pH indicator is phenol red and a concentration of phenol red in the base medium is about 5 mg/L.

In various embodiments, the salts are inorganic salts including calcium nitrate (Ca(NO₃)₂ 4H₂O), magnesium sulfate (MgSO₄) (anhyd.), potassium chloride (KCl), sodium bicarbonate (NaHCO₃), sodium chloride (NaCl), and sodium phosphate dibasic (Na₂HPO₄) anhydrous.

In various embodiments, a concentration of calcium nitrate (Ca(NO₃)₂ 4H₂O) in the base medium is about 50 mg/L, about 60 mg/L, about 70 mg/L, about 80 mg/L, about 90 mg/L, about 100 mg/L, about 110 mg/L, about 120 mg/L, about 130 mg/L, about 140 mg/L, or about 150 mg/L.

In various embodiments, a concentration of calcium nitrate (Ca(NO₃)₂ 4H₂O) in the base medium is about 100 mg/L.

In various embodiments, a concentration of magnesium sulfate (MgSO₄) (anhyd.) in the base medium is about 20 mg/L, about 30 mg/L, about 40 mg/L, about 50 mg/L, about 60 mg/L, about 70 mg/L, about 80 mg/L, about 90 mg/L, or about 100 mg/L.

In various embodiments, a concentration of magnesium sulfate (MgSO+) (anhyd.) in the base medium is about 48.84 mg/L.

In various embodiments, a concentration of potassium chloride (KC1) in the base medium is about 200 mg/L, about 300 mg/L, about 400 mg/L, about 500 mg/L, or about 600 mg/L.

In various embodiments, a concentration of potassium chloride (KCl) in the base medium is about 400 mg/L.

In various embodiments, a concentration of sodium bicarbonate (NaHCO₃) in the base medium is about 1,000 mg/L, about 1,500 mg/L, about 2,000 mg/L, about 2,500 mg/L, about 3,000 mg/L, about 3,500 mg/L, or about 4,000 mg/L.

In various embodiments, a concentration of sodium bicarbonate (NaHCO₃) in the base medium is about 2,000 mg/L.

In various embodiments, a concentration of sodium chloride (NaCl) in the base medium is about 3,000 mg/L, about 4,000 mg/L, about 5,000 mg/L, about 6,000 mg/L, about 7,000 mg/L, about 8,000 mg/L, about 9,000 mg/L, about 10,000 mg/L, about 11,000 mg/L, or about 12,000 mg/L.

In various embodiments, a concentration of sodium chloride (NaCl) in the base medium is about 6,000 mg/L.

In various embodiments, a concentration of sodium phosphate dibasic (Na₂HPO₄) anhydrous in the base medium is about 400 mg/L, about 500 mg/L, about 600 mg/L, about 700 mg/L, about 800 mg/L, about 900 mg/L, about 1,000 mg/L, about 1,100 mg/L, or about 1,200 mg/L.

In various embodiments, a concentration of sodium phosphate dibasic (Na₂HPO₄) anhydrous in the base medium is about 800 mg/L.

In various embodiments, the amino acids include reduced glutathione among others.

In various embodiments, the amino acids further include Glycine, L-Arginine, L-Asparagine, L-Aspartic acid, L-Cystine, L-Glutamic Acid, L-glutamine, L-Histidine, L-Hydroxyproline, L-Isoleucine, L-Leucine, L-Lysine hydrochloride, L-Methionine, L-Phenylalanine, L-Proline, L-Serine, L-Threonine, L-Tryptophan, L-Tyrosine disodium salt dehydrate, and L-Valine.

In various embodiments, concentrations of each amino acid are in a range of about 1-about 500 mg/L, about 100-about 400 mg/L, about 150-about 350 mg/L, or about 200-about 250 mg/L, a concentration of L-glutamine being the highest and a concentration of L-Tryptophan being the lowest among the concentrations of the amino acids.

In various embodiments, concentrations of each amino acid are in a range of about 1-about 500 mg/L, a concentration of L-glutamine being the highest and a concentration of L-Tryptophan being the lowest among the concentrations of the amino acids.

In various embodiments, the vitamins include biotin, choline chloride, D-calcium pantothenate, folic acid, niacinamide, para-aminobenzoic acid, pyridoxine hydrochloride, riboflavin, thiamine hydrochloride, vitamin B12, and i-inositol.

In various embodiments, concentrations of each vitamin are in a range of about 0.001-about 175 mg/L, about 0.005-about 150 mg/L, about 0.01-about 125 mg/L, about 0.05-about 100 mg/L, about 0.01-about 75 mg/L, or about 0.05-about 50 mg/L, a concentration of i-inositol being the highest and a concentration of vitamin B12 being the lowest among the concentrations of the vitamins.

In various embodiments, concentrations of each vitamin are in a range of about 0.001-about 175 mg/L.

In various embodiments, amounts of the recombinant human albumin and L-ascorbic acid 2-phosphate in the formulation are in ranges of about 100 μg-about 2500 μg/mL and about 45 μg-about 1,100 μg, respectively, per 500 mL of the base medium.

In various embodiments, the amounts of recombinant human albumin and L-ascorbic acid 2-phosphate in the formulation are in ranges of about 400 μg-about 600 μg/mL and about 150 μg-about 300 μg/mL, respectively, per 500 mL of the base medium.

In various embodiments, the amounts of recombinant human albumin and L-ascorbic acid 2-phosphate in the formulation are in ranges of about 450 μg-about 550 μg/mL and about 200 μg-about 250 μg/mL, respectively, per 500 mL of the base medium.

In various embodiments, amounts of insulin, heparin, and hydrocortisone in the formulation are in ranges of about 1 μg-about 25 μg/mL, about 5 μg-about 110 μg/mL, and about 0.05 μg-about 1 μg/mL, respectively, per 500 mL of the base medium.

In various embodiments, amounts of insulin, heparin, and hydrocortisone in the formulation are in ranges of about 3 μg-about 7 μg/mL, about 15 μg-about 30 μg/mL, and about 0.1 μg-about 0.5 μg/mL, respectively, per 500 mL of the base medium.

In various embodiments, amounts of insulin, heparin, and hydrocortisone in the formulation are in ranges of about 4 μg-about 6 μg/mL, about 20 μg-about 25 μg/mL, and about 0.2 μg-about 0.4 μg/mL, respectively, per 500 mL of the base medium.

In various embodiments, the synthetic analogue of IGF-I is LONG® R³-IGF-I (recombinant analog of human insulin-like growth factor-I (IGF-I), and an amount of LONG® R³-IGF-I in the formulation is in a range of about 4 ng-100 ng/ml, about 6 ng-80 ng/ml, about 8 ng-60 ng/ml, or about 10 ng-30 ng/ml per 500 mL of the base medium.

In various embodiments, the synthetic analogue of IGF-I is LONG® R³-IGF-I (recombinant analog of human insulin-like growth factor-I (IGF-I), and an amount of LONG® R³-IGF-I in the formulation is in a range of about 4 ng-100 ng/ml or about 10 ng-30 ng/mL per 500 mL of the base medium.

In various embodiments, amounts of VEGF, EGF, and bFGF in the formulation are in ranges of about 0.1 ng-about 2.5 ng/ml, about 1 ng-about 25 ng/ml, and about 2 ng-50 ng/mL, respectively, per 500 mL of the base medium.

In various embodiments, amounts of VEGF, EGF, and bFGF in the formulation are in ranges of about 0.3 ng-about 0.7 ng/ml, about 3 ng-about 7 ng/ml, and about 5 ng-15 ng/mL, respectively, per 500 mL of the base medium.

In various embodiments, amounts of VEGF, EGF, and bFGF in the formulation are in ranges of about 0.4 ng-about 0.6 ng/ml, about 4 ng-about 6 ng/ml, and about 8 ng-12 ng/mL, respectively, per 500 mL of the base medium.

In various embodiments, the formulation is cell culture media optimized for human induced pluripotent stem cell (hiPSC)-derived cardiovascular cell co-culture systems including 2D co-culture, cardiac organoids, and cardiac organ chips.

### Method of Growing Multiple Cardiovascular Cell Types Together Simultaneously

Various embodiments of the present invention provide for a method of growing multiple cardiovascular cell types in a device including multiple channels, wherein different types of cells are in different channels, the method comprising circulating a formulation through the multiple channels such that the different types of cells are grown in the formulation simultaneously without requiring different culture media in each channel having a different cell type, wherein the formulation includes a base medium comprising glucose, a pH indicator, salts, amino acids, and vitamins; recombinant human albumin; L-ascorbic acid 2-phosphate; and at least one or more of insulin, vascular endothelial growth factor (VEGF), epidermal growth factor (EGF), basic fibroblast growth factor (bFGF), a synthetic analogue of insulin-like growth factor (IGF)-I, heparin, or hydrocortisone.

In various embodiments, the base medium included in the formulation is Roswell Park Memorial Institute (RPMI) 1640 medium with L-glutamine.

In various embodiments, a concentration of glucose in the base medium is about 500 mg/L, about 1,000 mg/L, about 1,500 mg/L, about 2,000 mg/L, about 2,500 mg/L, about 3,000 mg/L, about 3,500 mg/L, or about 4,000 mg/L.

In various embodiments, a concentration of glucose in the base medium is about 2,000 mg/L.

In various embodiments, the pH indicator is phenol red and a concentration of phenol red in the base medium is about 1 mg/L, about 2 mg/L, about 3 mg/L, about 4 mg/L, about 5 mg/L, about 6 mg/L, about 7 mg/L, about 8 mg/L, about 9 mg/L, or about 10 mg/L.

In various embodiments, the pH indicator is phenol red and a concentration of phenol red in the base medium is about 5 mg/L.

In various embodiments, the salts included in the formulation are inorganic salts including calcium nitrate (Ca(NO₃)₂ 4H₂O), magnesium sulfate (MgSO+) (anhyd.), potassium chloride (KCl), sodium bicarbonate (NaHCO₃), sodium chloride (NaCl), and sodium phosphate dibasic (Na₂HPO₄) anhydrous.

In various embodiments, a concentration of calcium nitrate (Ca(NO₃)₂ 4H₂O), in the base medium is about 50 mg/L, about 60 mg/L, about 70 mg/L, about 80 mg/L, about 90 mg/L, about 100 mg/L, about 110 mg/L, about 120 mg/L, about 130 mg/L, about 140 mg/L, or about 150 mg/L.

In various embodiments, a concentration of calcium nitrate (Ca(NO₃)₂ 4H₂O) in the base medium is about 100 mg/L.

In various embodiments, a concentration of magnesium sulfate (MgSO₄) (anhyd.) in the base medium is about 20 mg/L, about 30 mg/L, about 40 mg/L, about 50 mg/L, about 60 mg/L, about 70 mg/L, about 80 mg/L, about 90 mg/L, or about 100 mg/L.

In various embodiments, a concentration of magnesium sulfate (MgSO+) (anhyd.) in the base medium is about 48.84 mg/L.

In various embodiments, a concentration of potassium chloride (KCl) in the base medium is about 200 mg/L, about 300 mg/L, about 400 mg/L, about 500 mg/L, or about 600 mg/L.

In various embodiments, a concentration of potassium chloride (KCl) in the base medium is about 400 mg/L.

In various embodiments, a concentration of sodium bicarbonate (NaHCO₃) in the base medium is about 1,000 mg/L, about 1,500 mg/L, about 2,000 mg/L, about 2,500 mg/L, about 3,000 mg/L, about 3,500 mg/L, or about 4,000 mg/L.

In various embodiments, a concentration of sodium bicarbonate (NaHCO₃) in the base medium is about 2,000 mg/L.

In various embodiments, a concentration of sodium chloride (NaCl) in the base medium is about 3,000 mg/L, about 4,000 mg/L, about 5,000 mg/L, about 6,000 mg/L, about 7,000 mg/L, about 8,000 mg/L, about 9,000 mg/L, about 10,000 mg/L, about 11,000 mg/L, or about 12,000 mg/L.

In various embodiments, a concentration of sodium chloride (NaCl) in the base medium is about 6,000 mg/L.

In various embodiments, a concentration of sodium phosphate dibasic (Na₂HPO₄) anhydrous in the base medium is about 400 mg/L, about 500 mg/L, about 600 mg/L, about 700 mg/L, about 800 mg/L, about 900 mg/L, about 1,000 mg/L, about 1,100 mg/L, or about 1,200 mg/L.

In various embodiments, a concentration of sodium phosphate dibasic (Na₂HPO₄) anhydrous in the base medium is about 800 mg/L.

In various embodiments, the amino acids included in the formulation include reduced glutathione among others.

In various embodiments, the amino acids included in the formulation further include Glycine, L-Arginine, L-Asparagine, L-Aspartic acid, L-Cystine, L-Glutamic Acid, L-glutamine, L-Histidine, L-Hydroxyproline, L-Isoleucine, L-Leucine, L-Lysine hydrochloride, L-Methionine, L-Phenylalanine, L-Proline, L-Serine, L-Threonine, L-Tryptophan, L-Tyrosine disodium salt dehydrate, and L-Valine.

In various embodiments, concentrations of each amino acid are in a range of about 1-about 500 mg/L, about 100-about 400 mg/L, about 150-about 350 mg/L, or about 200-about 250 mg/L, a concentration of L-glutamine being the highest and a concentration of L-Tryptophan being the lowest among the concentrations of the amino acids.

In various embodiments, concentrations of each amino acid are in a range of about 1-about 500 mg/L, a concentration of L-glutamine being the highest and a concentration of L-Tryptophan being the lowest among the concentrations of the amino acids.

In various embodiments, the vitamins included in the formulation include biotin, choline chloride, D-calcium pantothenate, folic acid, niacinamide, para-aminobenzoic acid, pyridoxine hydrochloride, riboflavin, thiamine hydrochloride, vitamin B12, and i-inositol.

In various embodiments, concentrations of each vitamin are in a range of about 0.001-about 175 mg/L, about 0.005-about 150 mg/L, about 0.01-about 125 mg/L, about 0.05-about 100 mg/L, about 0.01-about 75 mg/L, or about 0.05-about 50 mg/L, a concentration of i-inositol being the highest and a concentration of vitamin B12 being the lowest among the concentrations of the vitamins.

In various embodiments, concentrations of each vitamin are in a range of about 0.001-about 175 mg/L, a concentration of i-inositol being the highest and a concentration of vitamin B12 being the lowest among the concentrations of the vitamins.

In various embodiments, amounts of the recombinant human albumin and L-ascorbic acid 2-phosphate in the formulation are in ranges of about 100 μg-about 2500 μg/mL and about 45 μg-about 1,100 μg, respectively, per 500 mL of the base medium.

In various embodiments, the amounts of recombinant human albumin and L-ascorbic acid 2-phosphate in the formulation are in ranges of about 400 μg-about 600 μg/mL and about 150 μg-about 300 μg/mL, respectively, per 500 mL of the base medium.

In various embodiments, the amounts of recombinant human albumin and L-ascorbic acid 2-phosphate in the formulation are in ranges of about 450 μg-about 550 μg/mL and about 200 μg-about 250 μg/mL, respectively, per 500 mL of the base medium.

In various embodiments, amounts of insulin, heparin, and hydrocortisone in the formulation are in ranges of about 1 μg-about 25 μg/mL, about 5 μg-about 110 μg/mL, and about 0.05 μg-about 1 μg/mL, respectively, per 500 mL of the base medium.

In various embodiments, amounts of insulin, heparin, and hydrocortisone in the formulation are in ranges of about 3 μg-about 7 μg/mL, about 15 μg-about 30 μg/mL, and about 0.1 μg-about 0.5 μg/mL, respectively, per 500 mL of the base medium.

In various embodiments, amounts of insulin, heparin, and hydrocortisone in the formulation are in ranges of about 4 μg-about 6 μg/mL, about 20 μg-about 25 μg/mL, and about 0.2 μg-about 0.4 μg/mL, respectively, per 500 mL of the base medium.

In various embodiments, the synthetic analogue of IGF-I is LONG® R³-IGF-I, and an amount of LONG® R³-IGF-I in the formulation is in a range of about 4 ng-100 ng/mL, about 6 ng-80 ng/mL, about 8 ng-60 ng/ml, or about 10 ng-30 ng/ml per 500 mL of the base medium.

In various embodiments, the synthetic analogue of IGF-I is LONG® R3-IGF-I, and an amount of LONG® R³-IGF-I in the formulation is in a range of about 4 ng-100 ng/ml or about 10 ng-30 ng/ml per 500 mL of the base medium.

In various embodiments, amounts of VEGF, EGF, and bFGF in the formulation are in ranges of about 0.1 ng-about 2.5 ng/ml, about 1 ng-about 25 ng/ml, and about 2 ng-50 ng/ml, respectively, per 500 mL of the base medium.

In various embodiments, amounts of VEGF, EGF, and bFGF in the formulation are in ranges of about 0.3 ng-about 0.7 ng/mL, about 3 ng-about 7 ng/mL, and about 5 ng-15 ng/mL, respectively, per 500 mL of the base medium.

In various embodiments, amounts of VEGF, EGF, and bFGF in the formulation are in ranges of about 0.4 ng-about 0.6 ng/ml, about 4 ng-about 6 ng/ml, and about 8 ng-12 ng/ml, respectively, per 500 mL of the base medium.

In various embodiments, the formulation is cell culture media optimized for human induced pluripotent stem cell (hiPSC)-derived cardiovascular cell co-culture systems including 2D co-culture, cardiac organoids, and cardiac organ chips.

In various embodiments, induced pluripotent stem cell derived-endothelial cells (iPSC-ECs) are in a first channel among the multiple channels and induced pluripotent stem cell derived-cardiac cells (iPSC-CCs) are in a second channel among the multiple channels, respectively, or the iPSC-Ecs are in the second channel and the iPSC-CCs are in the first channel, respectively.

In various embodiments, the iPSC-Ecs and the iPSC-CCs are in different channels.

In various embodiments, the formulation is optimized for growth of both the iPSC-Ecs and the iPSC-CCs.

In various embodiments, the iPSC-CCs are induced pluripotent stem cell-derived cardiomyocytes (iPSC-CMs).

In various embodiments, induced pluripotent stem cell-derived cardiac fibroblasts (iPSC-CFs) are in the first or second channel among the multiple channels.

In various embodiments, the iPSC-ECs, the iPSC-CMs, and iPSC-CFs are in different channels.

In various embodiments, the formulation is optimized for growth of the iPSC-ECs, the iPSC-CMs, and the iPSC-CFs.

In various embodiments, the iPSC-Ecs are induced pluripotent stem cell derived-vascular endothelial cells (iPSC-VECs).

In various embodiments, the iPSC-CCs are induced pluripotent stem cell derived-cardiomyocytes (iPSC-CMs), induced pluripotent stem cell derived-fibroblast cells (iPSC-FCs), or induced pluripotent stem cell derived-smooth muscle cells (iPSC-SMCs).

In various embodiments, the iPSC-Ecs are induced pluripotent stem cell derived-vascular endothelial cells (iPSC-vECs), and the iPSC-CCs are induced pluripotent stem cell derived-cardiomyocytes (iPSC-CMs).

In various embodiments, the device is a microfluidic organ-on-chip.

In various embodiments, the iPSC-Ecs are human iPSC-Ecs (hiPSC-Ecs).

In various embodiments, the iPSC-CCs are human iPSC-CCs (hiPSC-CCs).

In various embodiments, the first and second channels include polydimethylciloxane.

In various embodiments, the first channel and the second channel are microfluidic channels.

### Method of Producing iPSC-EC/CM Organoids

Various embodiments of the present invention provide for a method of producing induced pluripotent stem cell (iPSC)-derived cardiac organoids containing iPSC-derived endothelial cells (iPSC-ECs) and iPSC-derived cardiomyocytes (iPSC-CMs), the method including harvesting iPSC-ECs and iPSC-CMs in a well plate under a sterile cell culture environment; resuspending the harvested iPSC-ECs and iPSC-CMs in a formulation to generate cardiac organoids; centrifuging the well plate to aggregate the organoids at a bottom of each well of the well plate; and culturing the organoids with the formulation, changing the formulation about three times a week, wherein the formulation is optimized for growth of both the iPSC-ECs and the iPSC-CCs, and wherein the formulation includes a base medium comprising glucose, a pH indicator, salts, amino acids, and vitamins; recombinant human albumin; L-ascorbic acid 2-phosphate; and at least one or more of insulin, vascular endothelial growth factor (VEGF), epidermal growth factor (EGF), basic fibroblast growth factor (bFGF), a synthetic analogue of insulin-like growth factor (IGF)-I, heparin, or hydrocortisone.

In various embodiments, the base medium included in the formulation is Roswell Park Memorial Institute (RPMI) 1640 medium with L-glutamine.

In various embodiments, a concentration of glucose in the base medium is about 500 mg/L, about 1,000 mg/L, about 1,500 mg/L, about 2,000 mg/L, about 2,500 mg/L, about 3,000 mg/L, about 3,500 mg/L, or about 4,000 mg/L.

In various embodiments, a concentration of glucose in the base medium is about 2,000 mg/L.

In various embodiments, the pH indicator is phenol red and a concentration of phenol red in the base medium is about 1 mg/L, about 2 mg/L, about 3 mg/L, about 4 mg/L, about 5 mg/L, about 6 mg/L, about 7 mg/L, about 8 mg/L, about 9 mg/L, or about 10 mg/L.

In various embodiments, the pH indicator is phenol red and a concentration of phenol red in the base medium is about 5 mg/L.

In various embodiments, the salts included in the formulation are inorganic salts including calcium nitrate (Ca(NO₃)₂ 4H₂O), magnesium sulfate (MgSO₄) (anhyd.), potassium chloride (KC1), sodium bicarbonate (NaHCO₃), sodium chloride (NaCl), and sodium phosphate dibasic (Na₂HPO₄) anhydrous.

In various embodiments, a concentration of calcium nitrate (Ca(NO₃)₂ 4H₂O), in the base medium is about 50 mg/L, about 60 mg/L, about 70 mg/L, about 80 mg/L, about 90 mg/L, about 100 mg/L, about 110 mg/L, about 120 mg/L, about 130 mg/L, about 140 mg/L, or about 150 mg/L.

In various embodiments, a concentration of calcium nitrate (Ca(NO₃)₂ 4H₂O) in the base medium is about 100 mg/L.

In various embodiments, a concentration of magnesium sulfate (MgSO₄) (anhyd.) in the base medium is about 20 mg/L, about 30 mg/L, about 40 mg/L, about 50 mg/L, about 60mg/L, about 70 mg/L, about 80 mg/L, about 90 mg/L, or about 100 mg/L.

In various embodiments, a concentration of magnesium sulfate (MgSO₄) (anhyd.) in the base medium is about 48.84 mg/L.

In various embodiments, a concentration of potassium chloride (KC1) in the base medium is about 200 mg/L, about 300 mg/L, about 400 mg/L, about 500 mg/L, or about 600 mg/L.

In various embodiments, a concentration of potassium chloride (KCl) in the base medium is about 400 mg/L.

In various embodiments, a concentration of sodium bicarbonate (NaHCO₃) in the base medium is about 1,000 mg/L, about 1,500 mg/L, about 2,000 mg/L, about 2,500 mg/L, about 3,000 mg/L, about 3,500 mg/L, or about 4,000 mg/L.

In various embodiments, a concentration of sodium bicarbonate (NaHCO₃) in the base medium is about 2,000 mg/L.

In various embodiments, a concentration of sodium chloride (NaCl) in the base medium is about 3,000 mg/L, about 4,000 mg/L, about 5,000 mg/L, about 6,000 mg/L, about 7,000 mg/L, about 8,000 mg/L, about 9,000 mg/L, about 10,000 mg/L, about 11,000 mg/L, or about 12,000 mg/L.

In various embodiments, a concentration of sodium chloride (NaCl) in the base medium is about 6,000 mg/L.

In various embodiments, a concentration of sodium phosphate dibasic (Na2HPO+) anhydrous in the base medium is about 400 mg/L, about 500 mg/L, about 600 mg/L, about 700 mg/L, about 800 mg/L, about 900 mg/L, about 1,000 mg/L, about 1,100 mg/L, or about 1,200 mg/L.

In various embodiments, a concentration of sodium phosphate dibasic (Na₂HPO₄) anhydrous in the base medium is about 800 mg/L.

In various embodiments, the amino acids included in the formulation include reduced glutathione among others.

In various embodiments, the amino acids included in the formulation further include Glycine, L-Arginine, L-Asparagine, L-Aspartic acid, L-Cystine, L-Glutamic Acid, L-glutamine, L-Histidine, L-Hydroxyproline, L-Isoleucine, L-Leucine, L-Lysine hydrochloride, L-Methionine, L-Phenylalanine, L-Proline, L-Serine, L-Threonine, L-Tryptophan, L-Tyrosine disodium salt dehydrate, and L-Valine.

In various embodiments, concentrations of each amino acid are in a range of about 1-about 500 mg/L, about 100-about 400 mg/L, about 150-about 350 mg/L, or about 200-about 250 mg/L, a concentration of L-glutamine being the highest and a concentration of L-Tryptophan being the lowest among the concentrations of the amino acids.

In various embodiments, concentrations of each amino acid are in a range of about 1-about 500 mg/L, a concentration of L-glutamine being the highest and a concentration of L-Tryptophan being the lowest among the concentrations of the amino acids.

In various embodiments, the vitamins included in the formulation include biotin, choline chloride, D-calcium pantothenate, folic acid, niacinamide, para-aminobenzoic acid, pyridoxine hydrochloride, riboflavin, thiamine hydrochloride, vitamin B12, and i-inositol.

In various embodiments, concentrations of each vitamin are in a range of about 0.001-about 175 mg/L, about 0.005-about 150 mg/L, about 0.01-about 125 mg/L, about 0.05-about 100 mg/L, about 0.01-about 75 mg/L, or about 0.05-about 50 mg/L, a concentration of i-inositol being the highest and a concentration of vitamin B12 being the lowest among the concentrations of the vitamins.

In various embodiments, concentrations of each vitamin are in a range of about 0.001-about 175 mg/L, a concentration of i-inositol being the highest and a concentration of vitamin B12 being the lowest among the concentrations of the vitamins.

In various embodiments, amounts of the recombinant human albumin and L-ascorbic acid 2-phosphate in the formulation are in ranges of about 100 μg-about 2500 μg/mL and about 45 μg-about 1,100 μg, respectively, per 500 mL of the base medium.

In various embodiments, the amounts of recombinant human albumin and L-ascorbic acid 2-phosphate in the formulation are in ranges of about 400 μg-about 600 μg/mL and about 150 μg-about 300 μg/mL, respectively, per 500 mL of the base medium.

In various embodiments, the amounts of recombinant human albumin and L-ascorbic acid 2-phosphate in the formulation are in ranges of about 450 μg-about 550 μg/mL and about 200 μg-about 250 μg/mL, respectively, per 500 mL of the base medium.

In various embodiments, amounts of insulin, heparin, and hydrocortisone in the formulation are in ranges of about 1 μg-about 25 μg/mL, about 5 μg-about 110 μg/mL, and about 0.05 μg-about 1 μg/mL, respectively, per 500 mL of the base medium.

In various embodiments, amounts of insulin, heparin, and hydrocortisone in the formulation are in ranges of about 3 μg-about 7 μg/mL, about 15 μg-about 30 μg/mL, and about 0.1 μg-about 0.5 μg/mL, respectively, per 500 mL of the base medium.

In various embodiments, amounts of insulin, heparin, and hydrocortisone in the formulation are in ranges of about 4 μg-about 6 μg/mL, about 20 μg-about 25 μg/mL, and about 0.2 μg-about 0.4 μg/mL, respectively, per 500 mL of the base medium.

In various embodiments, the synthetic analogue of IGF-I is LONG® R³-IGF-I, and an amount of LONG® R³-IGF-I in the formulation is in a range of about 4 ng-100 ng/ml, about 6 ng-80 ng/mL, about 8 ng-60 ng/ml, or about 10 ng-30 ng/ml per 500 mL of the base medium.

In various embodiments, the synthetic analogue of IGF-I is LONG® R³-IGF-I, and an amount of LONG® R³-IGF-I in the formulation is in a range of about 4 ng-100 ng/ml or about 10 ng-30 ng/mL per 500 mL of the base medium.

In various embodiments, amounts of VEGF, EGF, and bFGF in the formulation are in ranges of about 0.1 ng-about 2.5 ng/ml, about 1 ng-about 25 ng/ml, and about 2 ng-50 ng/mL, respectively, per 500 mL of the base medium.

In various embodiments, amounts of VEGF, EGF, and bFGF in the formulation are in ranges of about 0.3 ng-about 0.7 ng/ml, about 3 ng-about 7 ng/mL, and about 5 ng-15 ng/mL, respectively, per 500 mL of the base medium.

In various embodiments, amounts of VEGF, EGF, and bFGF in the formulation are in ranges of about 0.4 ng-about 0.6 ng/ml, about 4 ng-about 6 ng/ml, and about 8 ng-12 ng/ml, respectively, per 500 mL of the base medium.

In various embodiments, the formulation is cell culture media optimized for human induced pluripotent stem cell (hiPSC)-derived cardiovascular cell co-culture systems including 2D co-culture, cardiac organoids, and cardiac organ chips.

In various embodiments, the iPSC-ECs are induced pluripotent stem cell derived-vascular endothelial cells (iPSC-vECs).

In various embodiments, the iPSC-CCs are induced pluripotent stem cell derived-cardiomyocytes (iPSC-CMs), induced pluripotent stem cell derived-fibroblast cells (iPSC-FCs), or induced pluripotent stem cell derived-smooth muscle cells (iPSC-SMCs).

In various embodiments, the iPSC-ECs are induced pluripotent stem cell derived-vascular endothelial cells (iPSC-VECs).

In various embodiments, the iPSC-CCs are induced pluripotent stem cell derived-cardiomyocytes (iPSC-CMs).

In various embodiments, the iPSC-ECs are human iPSC-ECs (hiPSC-ECs).

In various embodiments, the iPSC-CCs are human iPSC-CCs (hiPSC-CCs).

In various embodiments, the harvesting iPSC-ECs and iPSC-CMs includes harvesting iPSC-ECs and iPSC-CMs in a ratio of 1:9.

In various embodiments, the harvesting iPSC-ECs and iPSC-CMs comprises harvesting about 2,000 or 2,000 iPSC-ECs and about 18,000 or 18,000 iPSC-CMs per organoid.

In various embodiments, the harvested iPSC-ECs and iPSC-CMs are resuspended in about 200 μL or 200 μL of the formulation in each well of the well plate such that cardiac organoids with about 20,000 or 20,000 cells are generated.

In various embodiments, each generated organoid has about 2,000 or 2,000 iPSC-ECs and about 18,000 or 18,000 iPSC-CMs.

In various embodiments, the harvesting iPSC-ECs and iPSC-CMs includes harvesting about 1,000 or 1,000 iPSC-ECs and about 9,000 or 9,000 iPSC-CMs per organoid.

In various embodiments, the harvested iPSC-ECs and iPSC-CMs are resuspended in about 200 μL or 200 μL of the formulation in each well of the well plate such that cardiac organoids with about 10,000 or 10,000 cells are generated.

In various embodiments, each generated organoid has about 1,000 or 1,000 iPSC-ECs and about 9,000 or 9,000 iPSC-CMs.

In various embodiments, the well plate is centrifuged for about 10 or 10 seconds at about 200× or 200× gravity to aggregate the organoids at the bottom of the wells.

In various embodiments, the aggregated organoids are cultured in the formulation at 37° C. under 5% CO₂ condition indefinitely.

In various embodiments, the changing the formulation three times a week comprises conducting a 75% media change of the formulation to prevent aspiration of the organoids from wells.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1—Heart Media Formulation

Heart Media is chemically defined, multi-lineage cardiac spheroid medium to grow hiPSC-CMs, hiPSC-ECs, and hiPSC-CFs as cardiac spheroids. To prepare the Heart Media, the components shown in the following table were added to 500 mL of RPMI 1640 with L-Glutamine (Gibco/ThermoFisher, Waltham, MA). The Heart Media may be supplemented as necessary with 20% fetal bovine serum only during initial plating process.

| Component | Concentration | |
|---|---|---|
| Recombinant human albumin (ScienCell Research | 505 | μg/mL |
| Laboratories, Cat. No. OsrHSA) | | |
| L-ascorbic acid 2-phosphate | 220 | μg/mL |
| (Fujifilm Wako Chemicals, Cat. | | |
| No. 323-44822) | | |
| Insulin | 5 | μg/mL |
| VEGF (Lonza, Cat. No. CC-4114A) | 0.5 | ng/mL |
| hEGF (Lonza, Cat. No. CC-4317A) | 5 | ng/mL |
| hFGF-B (Lonza, Cat. No. CC-4113A) | 10 | ng/mL |
| LONG R3-IGF-I (Lonza, Cat. No. CC-4115A) | 20 | ng/mL |
| Heparin (Lonza, Cat. No. CC-4396A) | 22.5 | μg/mL |
| Hydrocortisone (Lonza, Cat. No. CC-411A) | 0.2 | μg/mL |

### Example 2—hiPSC-EC/CM Organoid Production

hiPSC-derived cardiac organoids containing endothelial cells (hiPSC-ECs) and cardiomyocytes (hiPSC-CMs) were produced according to the following protocol:

1. Ensure that an ultra-low adhesion 96 well plate (e.g. corning costar 7007) is available. No extracellular matrix coating is needed. Ensure that appropriate volume of Heart Media is available for the organoid creation process.

2. In a sterile cell culture environment, harvest 2,000 hiPSC-ECs and 18,000 hiPSC-CMs per organoid. hiPSC-EC and CM production is described elsewhere and published.

3. Resuspend the cell amounts from the previous step in 200μL Heart Media per well, to create cardiac organoids with 20,000 cells. Each organoid should have 2,000 hiPSC-ECs and 18,000 hiPSC-CMs. Alternatively, organoids with the same ratio of EC: CM cell number can be created, with 10,000 cells total (1,000 ECs and 9,000 CMs) instead of 20,000.

4. Centrifuge the 96 well plate briefly (10 seconds) at 200× gravity to aggregate the organoids at the bottom of the well.

5. Culture at standard cell culture conditions (e.g. 37°° C., 5% CO₂) indefinitely with Heart Media changing Heart Media three times a week. Conduct a 75% media change to prevent aspiration of organoid from the well.

Organoids produced according to the above protocol should start beating within one week.

### Example 3—Growth of Cardiac Organoids

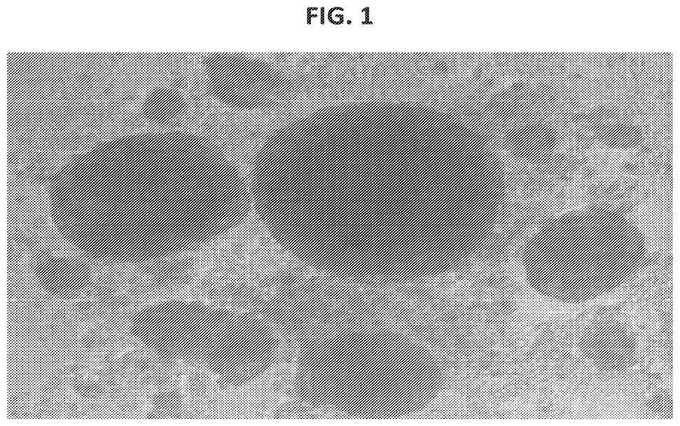

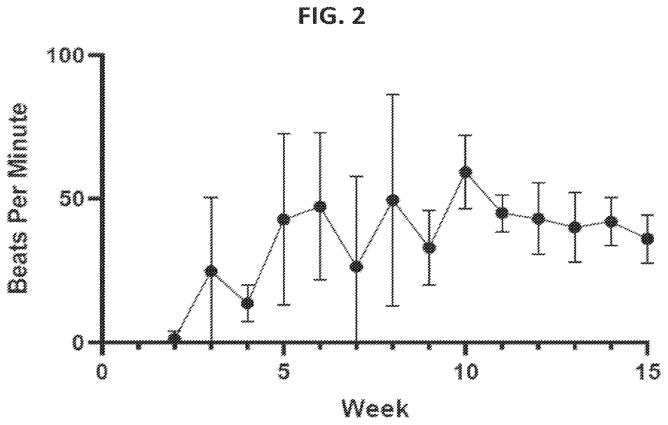

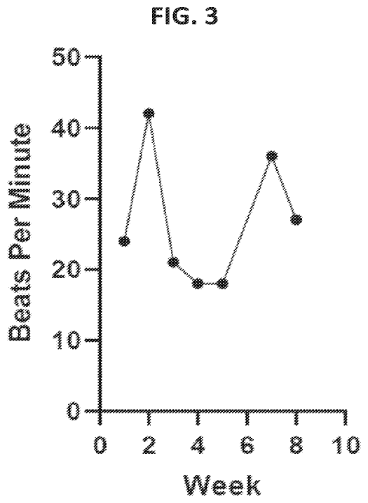

The cardiac organoids produced as described in Example 2 were grown in the Heart Media described in Example 1 for one week. FIG. 1 shows the cardiac organoids grown for one week. The cardiac organoids were composed of 90% hiPSC-derived cardiomyocytes and 10% hiPSC-derived endothelial cells. Further, FIG. 2 shows the beat rates for cardiac organoids grown in the Heart Media for 15 weeks. Furthermore, FIG. 3 shows beat rates of 2D hiPSC-CMS plated in the Heart Media for 9 weeks.

### Example 4—hiPSC-Cardiomyocyte/Endothelial Cell Co-Culture Functionality Data

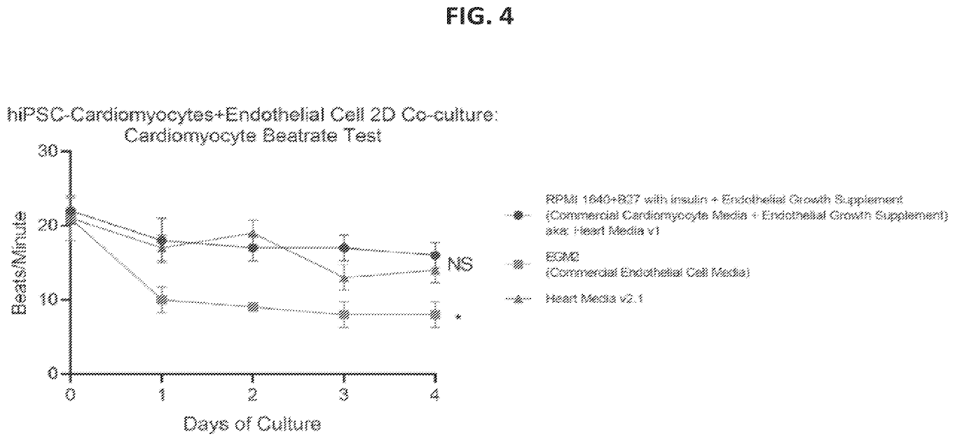

Cardiomyocyte beat rate tests were performed in hiPSC-Cardiomyocytes+Endothelial Cell 2D co-culture, using three different types of culture media, for co-culture samples 1, 2, and 3. Co-culture sample 1 was grown in RPMI 1640+B27 with insulin+Endothelial Growth Supplement (commercially available cardiomyocyte media, B27 supplement obtained from ThermoFisher, Waltham, MA mixed with Endothelia Growth Supplement obtained from Lonza Group, Basel, Switzerland). Co-culture sample 2 was grown in EGM2 (commercially available endothelial cell media obtained from Lonza Group, Basel, Switzerland). Co-culture sample 3 was grown in the Heart Media described in Example 1. As shown in FIG. 4, beats per minute obtained from sample 3 was clearly higher than beats per minute obtained from sample 2 throughout 4 days of culture. Further, beats per minute obtained from sample 3 was more or less similar to beats per minute obtained from sample 1. However, preparation of the culture media used for sample 1 required mixing the endothelial growth supplement with the commercially available cardiomyocyte media, and this, it was not cost effective.

### Example 5—hiPSC-Cardiomyocyte/Endothelial Cell Co-Culture Cell Viability Data

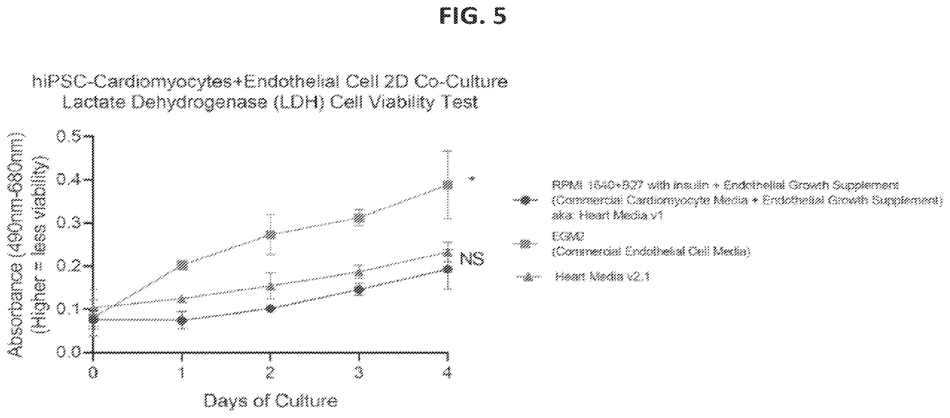

Lactate dehydrogenase (LDH) cell viability tests were performed in hiPSC-Cardiomyocytes+Endothelial Cell 2D co-culture, using three different types of culture media, for co-culture samples 1, 2, and 3 described in Example 4. As shown in FIG. 5, viability of sample 3 was clearly higher than viability of sample 2 throughout 4 days of culture. Further, although viability of sample 3 was a little lower than viability of sample 1 throughout 4 days of culture, the difference between them was not so great. That is, the Heart Media described in Example 1 is comparable with RPMI 1640+B27 with insulin +Endothelial Growth Supplement, which requires commercially available cardiomyocyte media, in terms of its efficiency in viability of co-cultured hiPSC-Cardiomyocytes and Endothelial cells.

### Example 6—Protocol for Maintenance and Expansion/Passage of hiPSCs

1. To prepare a 6-well plate coated with Matrigel extracellular matrix, thaw Matrigel at 4° C. and add 220 μL cold Matrigel to 12 mL DMEM, for a surface coverage of 0.25 μg/cm² (2X concentration per plate). Add 2 mL of Matrigel solution per well of a 6-well plate and incubate for at least 1 hour at 37° C. before use. Matrigel-coated plates can typically be kept at 37° C. for 2 weeks, if Matrigel solution does not evaporate.

2. Obtain a vial of hiPSCs (ideally below passage 30) from a frozen stock and thaw in hand at room temperature. Once thawing is nearly completed, add 1 mL hiPSC passaging medium to each vial and transfer the contents to a 15 ml conical tube to dilute the Cryostor. An ideal concentration would be 1 million cells per mL in each frozen hiPSC vial.

3. Centrifuge the cell suspension at 400 rcf for 5 minutes. Aspirate the supernatant completely and resuspend the cell pellet in 2 mL hiPSC passaging medium.

4. Remove Matrigel solution from a 6-well plate and plate hiPSCs suspended in 2 mL hiPSC passaging medium per well.

5. 24 hours after plating cells, replace the medium with 2 mL fresh mTeSR 1 maintenance medium. Incubate the cultures until they reach 80-100% confluence, then replace with mTeSR 1 medium daily. The optimal cell confluency for differentiation can vary between lines, and it is recommended to optimize confluency for the cell line.

6. Passage one well from a 6-well plate of hiPSCs, as follows to maintain the hiPSC line for future experiments and differentiations.

A. Remove mTeSR 1 maintenance medium from an 80-100% confluent well of hiPSCs and add 1 mL hiPSC dissociation buffer. Incubate the plate for 10 minutes at room temperature to partially dissociate the hiPSCs.

B. Gently aspirate the hiPSC dissociation buffer, ensuring cells are not completely dissociated. If examined under the microscope, the hiPSC colonies should be breaking apart into single cells and only lightly adhered to the cell culture plate.

C. Using a 1000-μL manual pipette, disturb the loosened hiPSCs with 1 mL fresh hiPSC passaging medium. Transfer the cell suspension to a new 15 ml conical tube and add 9 mL hiPSC passaging medium for a 1:10 dilution. The 1:10 dilution can be adjusted depending on the status of the starting confluency of the hiPSCs.

D. Obtain a new Matrigel-coated 6-well plate and aspirate the Matrigel solution. To each well, add 1 mL of the 1:10 hiPSCs suspension; shake the plate back and forth a few times to distribute cells evenly into the wells.

E. Culture the passaged hiPSCs at 37° C. and replace the medium daily with 2 mL mTeSR 1 maintenance medium for approximately 4 days. The culture should be ready to passage again in around 4 days when hiPSCs reach at least 80% confluence.

### Example 7—Protocol for Differentiation of hiPSCs into Cardiomyocytes

Induce Differentiation of hiPSCs into Cardiomyocytes—

1. After hiPSC passaging, from the remaining 5 wells of hiPSCs in a 6-well plate (step 5 in Example 6), remove the mTeSR 1 medium, and add 2 mL fresh mesoderm induction medium containing CHIR99021. Incubate the cells for 48 hours. Since the optimal CHIR99021 concentration for differentiation can vary between lines, it is recommended to optimize confluency and CHIR99021 concentration for the cell line.

2. After 48 hours of CHIR99021 treatment (day 2 of differentiation), aspirate the medium and replace with 2 mL fresh mesoderm induction medium. Incubate the cells for 24 hours.

3. After 24 hours (day 3 of differentiation), aspirate the medium and replace with 2 mL fresh mesoderm induction medium containing 2 μM Wnt-C59. Incubate the cells for 48 hours.

4. After 48 hours (day 5 of differentiation), aspirate the medium and replace with 2 mL fresh mesoderm induction medium. Incubate the cells for 48 hours.

5. After 48 hours (day 7 of differentiation), aspirate the medium and replace with 2 mL fresh cardiomyocyte maintenance medium. Incubate the cells for 72 hours, and then replace the medium with 2 mL fresh cardiomyocyte maintenance medium every 48 hours. beginning at day 10. Day 6-7 is typically the earliest time point that differentiated hiPSCs-CMs begin beating.

Metabolically Purify the hiPSC-CMs from Non-Cardiomyocytes—

6. Once cells begin beating, replace the medium with 2 mL cardiomyocyte purification medium every 48 hours, if necessary. Cardiomyocyte purification medium lacks glucose, leading to non-cardiomyocyte cell death after 24 hours. Cardiomyocytes may also stop beating and begin to die due to metabolic immaturity. Examine cardiomyocytes regularly to evaluate excess cell death.

7. Stop glucose starvation by replacing the medium with 2 mL cardiomyocyte maintenance medium after purification is complete or if hiPSC-CM death becomes excessive. The purified hiPSC-CMs are ready for use.

Immunofluorescence of hiPSC-CMs for Quality Control—

8. hiPSC-CMs can be replated from a 6-well plate post day 12 of differentiation to a cell culture chamber slide or additional plate format for quality control staining. The new plate format should be coated with double the Matrigel concentration, or 4 mg cold Matrigel to 12 mL DMEM (4X concentration). This concentration and volume can be scaled up or down depending on the size and need per plate. Cardiomyocyte passaging media should be prepared fresh for each replating. It is recommended to seed each chamber slide well with at least 200,000 hiPSC-CMs. Increasing the concentration of Matrigel from 2X to 4X will also help prevent hiPSC-CMs from peeling or beating off the new plate format.

9. Use primary antibodies anti-α-actinin and anti-cardiac troponin T at 1:200 dilutions in 3% BSA in PBS along with Alexa-Fluor-conjugated secondary antibodies at 1:500 dilutions in 3% BSA in PBS. α-actinin is a marker for α-cardiac muscle actinin. Cardiac troponin T is a marker for troponin T, the tropomyosin-binding subunit of troponin found in the heart.

### Example 8—Protocol for Differentiation of hiPSCs into Endothelial Cells

Induce Differentiation of hiPSCs into Endothelial Cells—

1. After passaging hiPSCs, from the remaining 5 wells of hiPSCs in a 6-well plate (step 5 in Example 6), remove the mTeSR 1 medium, and add 2 mL fresh mesoderm induction medium containing a high CHIR99021 concentration. Incubate the cells for 48 hrs. The “high” CHIR99021 concentration(s) for differentiation should range between 6-14 μM. It is recommended to optimize confluency and CHIR99021 concentration for the cell line.

2. After 48 hours of high CHIR99021 concentration treatment (day 2 of differentiation), aspirate the medium and replace with 2 mL fresh mesoderm induction medium containing 2 μM CHIR99021. Incubate the cells for 48 hrs. The concentration of CHIR99021 at 2 μM will serve as the “low” CHIR99021 concentration.

3. After 48 hours (day 4 of differentiation), aspirate the medium and replace with 2 mL fresh endothelial cell differentiation medium every 48 hours until day 12.

Harvest the hiPSC-ECs from hiPSCs—

4. To prepare a 24-well plate coated with 0.2% gelatin extracellular matrix, add 450 mL PBS and 50 mL 2% gelatin to a 500 mL glass media bottle. Autoclave the 0.2% gelatin solution on a standard liquid cycle for 30 minutes. Allow the solution to cool at room temperature. Add 1 mL of 0.2% gelatin per well of a 24-well plate and incubate for at least 1 hour at 37° C. before use. The 0.2% gelatin solution can be stored indefinitely at room temperature. Gelatin-coated plates can be kept at 37° C. for 2 weeks. Monitor plates to prevent solution evaporation.

5. At day 12 of differentiation, aspirate the medium and wash with 1 mL PBS. Aspirate the PBS and add 1.5 mL TrypLE Select for 5 minutes at 37° C.

6. After the 5 minute incubation, add 4.5 mL autoMACS running buffer to each well being sorted. Disturb the cells to single-cell suspension using a 1000-μL manual pipette. Transfer the cell suspension to a 15 mL conical tube. It is recommended to keep the autoMACS running buffer at 4° C. while sorting.

7. Pass the cells through a 30 um pre-separation filter into a new 15 mL conical tube to remove cell clumps. Moisten the filter with 1 mL autoMACS running buffer prior to adding the cells. One filter can usually withstand to filter 3-4 wells of cells from a 6-well plate.

8. For larger cell quantities, determine the cell number by diluting 10 μL cells into 10 μL 0.4% Trypan blue for a 1:2 dilution. It is recommended to limit the quantity of sorted cells to 10 million cells maximum.

9. Centrifuge the cell suspension at 400 rcf for 5 minutes. Aspirate the supernatant completely and resuspend the cell pellet in 80 μL autoMACS running buffer per 10 million cells. Add 20 μL CD144 microbeads per 10 million cells. Mix and incubate the 15 ml conical tube at 4° C. for 15 minutes. The volume of autoMACS running buffer and CD144 microbeads can be doubled depending on the total number of cells.

10. After the 15 minute incubation, wash cells by adding 1-2 mL autoMACS running buffer per 10 million cells. Centrifuge the cells at 400 rcf for 5 minutes and aspirate the supernatant completely.

11. Resuspend cells in 500 μL autoMACS running buffer. It is recommended to increase the volume of autoMACS running buffer by 500 μL per every 100,000 cells.

Magnetically Separate the hiPSC-ECs from Non-Endothelial Cells—

12. Place an LS column in the magnetic field of a suitable MACs separator. It is recommended to use one LS column for about 3 wells of cells from a 6-well plate. Scale up the amount of LS columns as necessary.

13. Prepare each LS column by rinsing with 3 mL autoMACs running buffer. Position an open 50 mL conical tube below each column to collect flow-through.

14. Apply the cell suspension obtained in step 11 onto the LS column and continue to collect the flow-through of unlabeled cells using an open 50 mL conical tube.

15. Wash each column three times with 3 mL autoMACS running buffer, continuing to collect the flow-through of unlabeled cells in the 50 ml conical tube.

16. In a quick manner, remove the LS column from the separator and place it in a 15 ml conical tube. Add 5 mL of autoMACs running buffer onto the column. Immediately following the buffer addition, flush out the magnetically labeled cells by pushing the plunger firmly into the column.

17. To increase the purity of the CD144⁺ cells, enrich the eluted fraction in step 16 through a new LS column positioned in the separator. Repeat the magnetic separation procedure as outlined in steps 12-16.

18. Following the completion of the second magnetic separation, centrifuge the eluted fraction in the 15 ml conical tube at 400 rcf for 5 minutes. Resuspend the pellet in 500 μL endothelial cell maintenance medium per well plating on the 0.2% gelatin-coated 24-well plate. It is recommended to plate 2-3 wells of cells from the original 6-well Matrigel plate into one well on the 0.2% gelatin-coated 24-well plate. This helps ensure the hiPSC-ECs will proliferate in a confluent manner.

19. Add 500 μL hiPSC-ECs per well on the 0.2% gelatin-coated 24-well plate for a passage 0 of sorted C144⁺hiPSC-ECs.

Maintain and Expand the hiPSC-ECs—

20. Following the hiPSC-EC sort on day 12, aspirate the medium and replace with 2 mL endothelial cell maintenance medium. Incubate the cells for 48 hours. Continue refreshing the medium with 2 mL endothelial cell maintenance medium every 48 hours until hiPSC-ECs reach confluence in each well.

21. Once at confluency, aspirate the medium and wash with 1 mL PBS. Aspirate the PBS and add 500 μL TrypLE Select for 5 minutes at 37° C.

22. After the 5 minute incubation, add 500 μL endothelial cell maintenance medium to each well. Disturb the cells to single-cell suspension using a 1000-μL manual pipette. Transfer the cell suspension to a 15 ml conical tube.

23. Centrifuge the cell suspension at 400 rcf for 5 minutes. Aspirate the supernatant completely and resuspend the cell pellet in 500 μL endothelial cell maintenance medium per well for a 24-well plate re-seeding. It is recommended to passage each well on the 24-well 0.2% gelatin plate at a 1:3 dilution.

24. Add 500 μL of the single cell solution to each well of the 0.2% gelatin-coated 24-well plate for a passage 1 of the sorted C144⁺hiPSC-ECs.

25. Following the hiPSC-EC passage, aspirate the medium and replace with 2 mL endothelial cell maintenance medium. Incubate the cells for 48 hours. Continue refreshing the medium with 2 mL endothelial cell maintenance medium every 48 hours until hiPSC-ECs reach confluence in each well.

26. Once at confluency, aspirate the medium and wash with 1 mL PBS. Aspirate the PBS and add 500 μL TrypLE Select for 5 minutes at 37° C.

27. After the 5 minute incubation, add 500 μL endothelial cell maintenance medium to each well. Disturb the cells to single-cell suspension using a 1000-μL manual pipette. Transfer the cell suspension to a 15 ml conical tube.

28. Centrifuge the cell suspension at 400 rcf for 5 minutes. Aspirate the supernatant completely and resuspend the cell pellet in 500 μL endothelial cell maintenance medium per well plating on the 0.2% gelatin-coated 24-well plate. It is recommended to passage each well on the 24-well 0.2% gelatin plate at a 1:3 dilution.

29. Add 500 μL hiPSC-ECs per well on the 0.2% gelatin-coated 24-well plate. This hiPSC-EC passage is passage 2.

Freeze the hiPSC-ECs—

30. After passage 2, the hiPSC-ECs can either be maintained with endothelial cell maintenance medium in culture or frozen for indefinite storage. If freezing, aspirate the medium from a well of the 24-well plate containing cells and add 1 mL TrypLE Express. Incubate the plate for 10 minutes at 37° C.

31. After the incubation at 37° C., add 2 mL endothelial cell maintenance medium to the well to deactivate the TrypLE Express. Disturb the cells to single-cell suspension and transfer to a 15 mL conical tube using a 1000-μL manual pipette.

32. Centrifuge the cell suspension at 400 rcf for 5 minutes. Aspirate the supernatant completely and resuspend the cell pellet in 1 mL of 4° C. cold Cryostor solution. It is recommended to add 1 mL of 500,000 hiPSC-ECs per vial at passage 2.

33. Distribute 1 mL to each freezing vial and transfer to a freezing Styrofoam vessel. Incubate the freezing vessel at −80° C. for 24 hours.

34. After the −80° C. incubation, transfer each vial to a liquid nitrogen cryotank for indefinite storage.

sImmunofluorescence of hiPSC-ECs for Quality Control—

35. hiPSC-ECs can be replated from a 24-well plate post passage 2 of differentiation to or thawed into a chamber slide or additional plate format coated with 0.2% gelatin for quality control staining. Endothelial cell maintenance medium with 20% FBS should be prepared fresh for each replating. It is recommended to seed each chamber slide well with at least 200,000 hiPSC-ECs.

36. Use primary antibodies CD31 (PECAM-1) and VE-cadherin at 1:200 dilutions along with Alex-Fluor-conjugated secondary antibodies at 1:500 dilutions. CD31 (PECAM-1) is a marker for CD31, a member of cell adhesion molecules expressed by endothelial cells. VE-cadherin is a marker for signaling, expression, and localization in endothelial cells.

### Example 9—Protocol for Differentiation of hiPSCs into Cardiac Fibroblasts

Induce Differentiation of hiPSCs into Cardiac Fibroblasts—

1. After passaging hiPSCs, from the remaining 5 wells of hiPSCs in a 6-well plate (step 5 in Example 6), remove the mTeSR 1 medium and add 2 mL fresh mesoderm induction medium containing a high CHIR99021 concentration. Incubate the cells for 48 hours. The “high” CHIR99021 concentration(s) for differentiation should range between 8-10 μM. It is recommended to optimize confluency and CHIR99021 concentration for the cell line.

2. After 48 hours of high CHIR99021 concentration treatment (day 2 of differentiation), aspirate the medium and replace with 2 mL fresh mesoderm induction medium. Incubate the cells for 24 hours.

3. After 24 hours (day 3 of differentiation), aspirate the medium and replace with 2 mL fresh mesoderm induction medium containing 2 μM Wnt-C59. Incubate the cells for 48 hours.

Maintain the hiPSC-CFs—

4. After 48 hours (day 5 of differentiation), aspirate the medium and replace with 2 mL fresh cardiac fibroblast differentiation medium every 48 hours until hiPSC-CF confluency is reached per well. hiPSC-CF confluence is usually reached by day 11 or 12 of differentiation but can differ per cell line.

Freeze the hiPSC-CFs—

5. The hiPSC-CFs can either be maintained with fibroblast differentiation medium in culture or frozen for indefinite storage. If freezing, aspirate the medium and add 1 mL TrypLE Select. Incubate the plate for 10 minutes at 37° C.

6. After the incubation at 37° C., add 2 mL fibroblast differentiation medium to deactivate the TrypLE Express. Disturb the cells to single-cell suspension and transfer to a 15 mL conical tube using a 1000-μL manual pipette.

7. Centrifuge the cell suspension at 400 rcf for 5 minutes. Aspirate the supernatant completely and resuspend the cell pellet in 1 mL 4° C. cold Cryostor.

8. Distribute 1 mL to each freezing vial and transfer to a freezing Styrofoam vessel. Incubate the freezing vessel at −80° C. for 24 hours.

9. After the −80° C. incubation, transfer each vial to a liquid nitrogen cryotank for indefinite storage.

Immunofluorescence of hiPSC-CFs for Quality Control—

10. hiPSC-CFs can be replated from a 6-well plate post differentiation or thawed into a chamber slide or additional plate format coated with a 2X Matrigel concentration for quality control staining. Cardiac fibroblast maintenance medium should be prepared fresh for each replating. It is recommended to seed each chamber slide well with at least 200,000 hiPSC-CFs.

11. Use primary antibodies vimentin and α-smooth muscle actin at 1:200 dilutions along with Alex-Fluor-conjugated secondary antibodies at 1:500 dilutions. Vimentin is a marker for actin filaments, a member of cell adhesion molecules. α-smooth muscle actin is a marker for actin proteins.

### Example 10—Protocol for Production of hiPSC-derived Cardiac Spheroids

Dissociate hiPSC-CMs from a 6-Well Plate—1. Prepare chemically defined, multi-lineage cardiac spheroid medium (heart

medium) supplemented with 20% FBS. 20% FBS should be added to the heart media for the initial plating process. It improves hiPSC-CM survival post-dissociation.

2. Obtain a 6-well plate containing purified population of hiPSC-CMs. Select the number of wells required for passaging. Wash each well of cells with 1 mL PBS. We have noticed that hiPSC-CM dissociation is easier in younger cells, ideally no older than day 20 post-differentiation.

3. Aspirate the PBS and add 2 mL TrypLE Select 10X into the selected well(s) of hiPSC-CMs. Incubate for 10 minutes at 37° C. During the 10 minute incubation, tap and shake the bottom of the wells selected for passaging every 2 minutes to help lift the cells.

4. After the 10 minute incubation, add 2 mL heart medium to each selected well to inactivate TrypLE Select 10X. Immediately dissociate hiPSC-CMs from the cell culture plate using a 1000-μL manual pipette by rigorously pipetting 50 times to prepare a single-cell suspension.

5. Transfer the cardiomyocyte suspension to a 15 ml conical tube. Centrifuge the cell suspension at 400 rcf for 5 minutes. Aspirate the supernatant and suspend the hiPSC-CMs in 1-2 mL heart medium depending on the pellet size.

Thaw hiPSC-ECs from a Cryovial—

6. Obtain a vial of hiPSC-ECs from a frozen stock and thaw in hand at room temperature. Repeat if multiple vials of hiPSC-ECs.

7. Once thawing is nearly completed, add 1 mL heart media to each vial and transfer the contents to a 15 ml conical tube. Add an additional 5 mL heart media to the 15 ml conical tube to dilute the cryopreservative.

8. Centrifuge the cell suspension at 400 rcf for 5 minutes. Aspirate the supernatant and suspend the hiPSC-ECs in 1-2 mL heart medium depending on the pellet size.

Thaw hiPSC-CFs from a Cryovial—

9. Obtain a vial of hiPSC-CFs from a frozen stock and thaw in hand at room temperature. Repeat if multiple vials of hiPSC-CFs.

10. Once thawing is nearly completed, add 1 mL heart media to each vial and transfer the contents to a 15 ml conical tube. Add an additional 5 mL heart media to the 15 mL conical tube to dilute the cryopreservative.

11. Centrifuge the cell suspension at 400 rcf for 5 minutes. Aspirate the supernatant and suspend the hiPSC-CFs in 1-2 mL heart medium depending on the pellet size.

Count the Total Number of hiPSC-CMs, hiPSC-ECs, and hiPSC-CFs—

12. Per each cell type, dilute 10 μL cells into 10 μL 0.4% Trypan blue for a 1:2 dilution. Pipette repeatedly to mix the cells and dye and draw up 10 μL to add to a hemocytometer.

13. Count all four quadrants of dispersed cells on a hemocytometer and record the total number of living cells.

14. Determine the amount (μL) of each cell type that is required to plate the total number of spheroids necessary, each at the ratio of 80% hiPSC-CMs, 10% hiPSC-ECs, and 10% hiPSC-CFs. The spheroid cellular ratio was adapted from Giacomelli et al 2020, Cell Stem Cell. It was also determined that a total of 20,000 cells (16,000 hiPSC-CMs, 2,000 hiPSC-ECs, and 2,000 hiPSC-CFs) plated in 200 μL heart media per spheroid was the ideal size distribution for quick and successful self-aggregation.

15. Combine the calculated amount of cells in a new 15 ml conical tube. Determine and add the remaining amount (μL) of Heart Media required to reach the total volume of 200 μL per spheroid formation.

16. Add 200 μL of the multi-cell suspension to each well of a clear, low adhesion U-bottom 96-well plate with no prior extracellular matrix coating. It is recommended to plate one spheroid per well.

17. Allow spheroids to begin self-assembly for 48 hours at 37° C.

18. As the 3D sphere-shape begins to form, perform 100 μL 50% media changes on the spheroids using 37° C. warm Heart Media every 48 hours. We have found that it usually takes 1-2 weeks for the spheroids to become full 3D spheres and start contracting.

### Example 11—Results

CM/EC/CF organoids showed improvements overall in structure, function, and metabolism in comparison to other cell type combinations. Structurally, sarcomere length and organization increased (characteristics of more mature CMs). Functionally, mechanical contraction showed increased duration and amplitude, calcium handling and electrophysiology were improved. With metabolism, mitochondrial respiration was increased. Emphasis was on tri-cellular crosstalk.

The following is attempted conditions for CM/EC/CF organoids:
Per organoid: 80% CMs+10% ECs+10% CFs;
20k cells: 16,000 CMs+2,000 ECs+2,000 CFs;
U-bottom 96-well plates;
Heart Media+20% FBS;
200 μL for 20 k ratio; and
No centrifuge.

FIG. l shows average beat rates of each CM/EC/CF organoid seed. Referring to

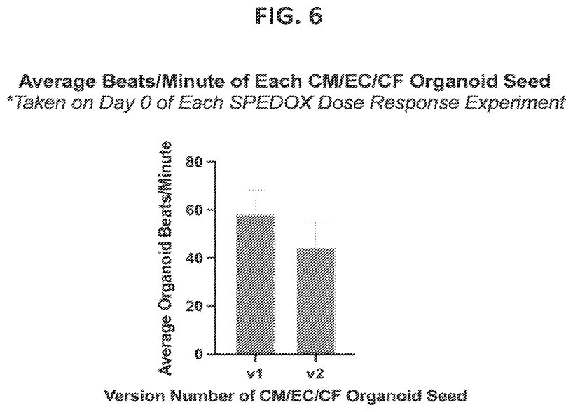

FIG. 6, the average beats/minute of v1 was 57.9 for 27 organoids and the average beats/minute of v2 was 44.4 for 24 organoids.

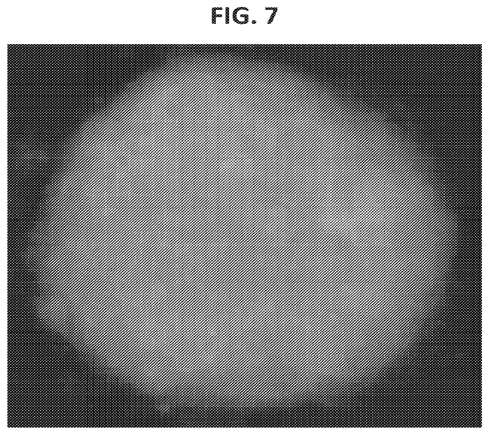

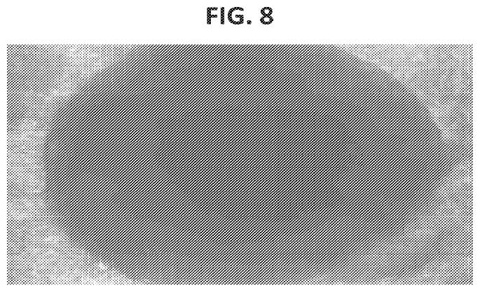

FIGS. 7 and 8 show images of CM/EC/CF organoids. FIG. 8 shows an immune-fluorescence image including ACTN2-GFP hiPSC-CMs, TUBA1_mTagRFPI hiPSC-ECs and 02iCTR hiPSC-CFs. FIG. 8 shows a brightfield image including ACTN2-GFP hiPSC-CMs, TUBA1_mTagRFPI hiPSC-ECs and 02iCTR hiPSC-CFs. Example 12-Reagents and Solutions

hiPSC dissociation buffer (to gently loosen hiPSCs from a culture plate) containing Phosphate-buffered saline (PBS; ThermoFisher Scientific, Cat. No. 14-190-136) (500 mL) and 0.5 M EDTA (ThermoFisher Scientific, Cat. No. 15-575-020) (500 μL).

hiPSC' passaging medium (to passage hiPSCs) containing mTESR-1 feeder-free pluripotent stem cell maintenance medium for culturing human ES and iPS cells (Stemcell Technologies, Cat. No. 85850) (500 mL) and 10 μM rho kinase inhibitor (ROCKi; Reprocell, Cat. No. 04-0012-02).

Mesoderm induction media (days 0 to 7 of hiPSC-CM differentiation; days 0 to 4 of hiPSC-EC differentiation; days 0 to 5 of hiPSC-CF differentiation) containing RPMI 1640 mammalian cell culture medium (ThermoFisher Scientific, Cat. No. 11875093) (500 mL) and 1× B27 supplement without insulin (ThermoFisher Scientific, Cat. No. A1895601) (10 mL), supplementing as necessary with Wnt-regulating small molecules—e.g., CHIR99021 GSK3-beta inhibitor (Cayman Chemical, Cat. No. 13122) or Wnt-C59 (ThermoFisher, Cat. No. 51-481-0)-from 10 mM stocks dissolved in DMSO.

Cardiomyocyte maintenance medium (after day 7 of hiPSC-CM differentiation) containing RPMI 1640 medium (500 mL) and 1× B27 supplement with insulin (ThermoFisher Scientific, Cat. No. 17504-044) (10 mL).

('ardiomyocyte passaging medium (to dissociate and plate differentiated hiPSC-CMs) containing RPMI 1640 medium (500 mL); 1× B27 supplement with insulin (10 mL); 20% fetal bovine serum (FBS; ThermoFisher Scientific, Cat. No. 10437-028); 1:100 antibiotic antimycotic (PSA; Corning, Cat. No. MT30004CI); 20 μg/mL insulin spike (ThermoFisher Scientific, Cat. No. 12585014); and 10 μM rho kinase inhibitor.

Cardiomyocyte purification medium (to metabolically select differentiated hiPSC-CMs) containing RPMI 1640 medium without glucose (ThermoFisher Scientific, Cat. No. 11879-020) (500 mL); 1×B27 supplement with insulin (10 mL); and Sodium DL-lactate solution (Millipore Sigma, Cat. No. L7900-100mL) (500 μL).

Endothelial cell differentiation medium (days 4 to 12 of hiPSC-EC differentiation) containing Endothelial cell growth media (Lonza, Cat. No. CC-3162) (500 mL); 50 ng/ml recombinant human VEGF165 (Peprotech, Cat. No. 100-20); 10 μM SB431542 (Cayman Chemicals, Cat. No. 13031); and 25 ng/ml human FGF-basic (Peprotech, Cat. No. 100-18B-1 MG).

Endothelial cell maintenance medium (after day 12 of hiPSC-EC differentiation) containing Endothelial cell growth media; 50 ng/mL recombinant human VEGF165; and 10 μM SB431542.

Cardiac fibroblast differentiation medium (after day 5 of hiPSC-CF differentiation until freezing) containing Endothelial cell growth media (Lonza, Cat. No. CC-3162) (500 mL) and 25 ng/mL human FGF-basic (Peprotech, Cat. No. 100-18B-1 MG).

Cardiac fibroblast maintenance medium (after thawing differentiated hiPSC-CFs) containing Advanced DMEM/F-12 (ThermoFisher Scientific, Cat. No. 12-634-010) (500 mL); 20% fetal bovine serum; 1% antibiotic antimycotic; 25 ng/mL human FGF-basic; and 10 μM rho kinase inhibitor.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A formulation comprising:
a base medium comprising glucose, a pH indicator, salts, amino acids, and vitamins;
recombinant human albumin;
L-ascorbic acid 2-phosphate; and
at least one or more of insulin, vascular endothelial growth factor (VEGF), epidermal growth factor (EGF), basic fibroblast growth factor (bFGF), a synthetic analogue of insulin-like growth factor (IGF)-I, heparin, or hydrocortisone.

2. The formulation of claim 1, wherein the base medium is Roswell Park Memorial Institute (RPMI) 1640 medium with L-glutamine.

3. The formulation of claim 1, wherein a concentration of glucose in the base medium is about 2,000 mg/L, and/or the pH indicator comprises phenol red and a concentration of phenol red in the base medium is about 5 mg/L.

4. (canceled)

5. The formulation of claim 3, wherein the salts are inorganic salts comprising calcium nitrate (Ca(NO₃)₂ 4H₂O), magnesium sulfate (MgSO₄) (anhyd.), potassium chloride (KCl), sodium bicarbonate (NaHCO₃), sodium chloride (NaCl), and sodium phosphate dibasic (Na₂HPO₄) anhydrous, and/or concentrations of calcium nitrate (Ca(NO₃)₂ 4H₂O), magnesium sulfate (MgSO₄) (anhyd.), potassium chloride (KCl). sodium bicarbonate (NaHCO₃), sodium chloride (NaCl), and sodium phosphate dibasic (Na₂HPO₄) anhydrous in the base medium are about 100 mg/L, about 48.84 mg/L, about 400 mg/L, about 2,000 mg/L, about 6,000 mg/L, and about 800 mg/L, respectively.

6. (canceled)

7. The formulation of claim 3, wherein the amino acids comprise reduced glutathione.

8. The formulation of claim 7, wherein the amino acids further comprise Glycine, L-Arginine, L-Asparagine, L-Aspartic acid, L-Cystine, L-Glutamic Acid, L-glutamine, L-Histidine, L-Hydroxyproline, L-Isoleucine, L-Leucine, L-Lysine hydrochloride, L-Methionine, L-Phenylalanine, L-Proline, L-Serine, L-Threonine, L-Tryptophan, L-Tyrosine disodium salt dehydrate, and L-Valine.

9. The formulation of claim 8, wherein a concentration of each amino acid is in a range of about 1-about 500 mg/L, a concentration of L-glutamine being the highest and a concentration of L-Tryptophan being the lowest among the concentrations of the amino acids.

10. The formulation of claim 8, wherein the vitamins comprise biotin, choline chloride, D-calcium pantothenate, folic acid, niacinamide, para-aminobenzoic acid, pyridoxine hydrochloride, riboflavin, thiamine hydrochloride, vitamin B12, and i-inositol, and/or a concentration of each vitamin is in a range of about 0.001-about 175 mg/L, a concentration of i-inositol being the highest and a concentration of vitamin B12 being the lowest among the concentrations of the vitamins.

11. (canceled)

12. The formulation of claim 1, wherein amounts of the recombinant human albumin and L-ascorbic acid 2-phosphate in the formulation are in ranges of about 100 μg-about 2500 μg/mL and about 45 μg-about 1,100 μg, respectively, per 500 mL of the base medium or in ranges of about 400 μg-about 600 μg/mL and about 150 μg-about 300 μg/mL, respectively, per 500 mL of the base medium;
wherein amounts of insulin, heparin, and hydrocortisone in the formulation are in ranges of about 1 μg-about 25 μg/mL, about 5 μg-about 110 μg/mL, and about 0.05 μg-about 1 μg/mL, respectively, per 500 mL of the base medium or in ranges of about 3 μg-about 7 μg/mL, about 15 μg-about 30 μg/mL, and about 0.1 μg-about 0.5 μg/mL, respectively, per 500 mL of the base medium;
wherein the synthetic analogue of IGF-I is LONG® R³-IGF-I, and an amount of LONG® R³-IGF-I in the formulation is in a range of about 4 ng-100 ng/mL or about 10 ng-30 ng/mL per 500 mL of the base medium; and/or
wherein amounts of VEGF, EGF, and bFGF in the formulation are in ranges of about 0.1 ng-about 2.5 ng/ml, about 1 ng-about 25 ng/ml, and about 2 ng-50 ng/ml, respectively, per 500 mL of the base medium or in ranges of about 0.3 ng-about 0.7 ng/mL. about 3 ng-about 7 ng/ml, and about 5 ng-15 ng/ml, respectively, per 500 mL of the base medium.

13. (canceled)

14. (canceled)

15. (canceled)

16. (canceled)

17. (canceled)

18. (canceled)

19. The formulation of claim 1, wherein the formulation is cell culture media optimized for human induced pluripotent stem cell (hiPSC)-derived cardiovascular cell co-culture systems including 2D co-culture, cardiac organoids, and cardiac organ chips.

20. A method of growing multiple cardiovascular cell types in a device comprising multiple channels, wherein different types of cells are in different channels, the method comprising:
circulating the formulation of claim 1 through the multiple channels such that the different types of cells are grown in the formulation simultaneously without requiring different culture media in each channel having a different cell type.

21. The method of claim 20, wherein:
induced pluripotent stem cell-derived endothelial cells (iPSC-ECs) and induced pluripotent stem cell-derived cardiac cells (iPSC-CCs) are in a first channel and a second channel among the multiple channels, or in the second channel and the first channel, respectively;
the iPSC-ECs and the iPSC-CCs are in different channels; and
the formulation is optimized for growth of both the iPSC-ECs and the iPSC-CCs.

22. The method of claim 21, wherein:
the iPSC-CCs are induced pluripotent stem cell-derived cardiomyocytes (iPSC-CMs), or the iPSC-ECs are induced pluripotent stem cell derived-vascular endothelial cells (iPSC-VECs);
induced pluripotent stem cell-derived cardiac fibroblasts (iPSC-CFs) are in the first or second channel among the multiple channels, or the iPSC-ECs, the iPSC-CMs, and iPSC-CFs are in different channels, or the iPSC-CCs are induced pluripotent stem cell derived-cardiomyocytes (iPSC-CMs), induced pluripotent stem cell-derived fibroblast cells (iPSC-FCs), or induced pluripotent stem cell-derived smooth muscle cells (iPSC-SMCs); and
the formulation is optimized for growth of the iPSC-ECs, the iPSC-CMs, and the iPSC-CFs.

23. (canceled)

24. The method of claim 21, wherein:
the iPSC-ECs are induced pluripotent stem cell-derived vascular endothelial cells (iPSC-vECs); and the iPSC-CCs are induced pluripotent stem cell-derived cardiomyocytes (iPSC-CMs).

25. (canceled)

26. The method of claim 22, wherein the iPSC-ECs are human iPSC-ECs (hiPSC-ECs), and/or wherein the iPSC-CFs are human iPSC-CFs (hiPSC-CFs).

27. (canceled)

28. The method of claim 21, wherein the device is a microfluidic organ-on-chip, wherein the first and second channels comprise polydimethylciloxane, or the different channels comprise polydimethylciloxane, and/or wherein the first channel and the second channel are microfluidic channels, or the different channels are microfluidic channels.

29. (canceled)

30. A method of producing induced pluripotent stem cell (iPSC)-derived cardiac organoids containing iPSC-derived endothelial cells (iPSC-ECs) and iPSC-derived cardiac cells (iPSC-CCs), the method comprising:
harvesting iPSC-ECs and iPSC-CCs in a well plate under a sterile cell culture environment;
resuspending the harvested iPSC-ECs and iPSC-CCs in the formulation of claim 1 to generate cardiac organoids;
centrifuging the well plate to aggregate the organoids at a bottom of each well of the well plate; and
culturing the organoids with the formulation, changing the formulation three times a week,
wherein the formulation is optimized for growth of both the iPSC-ECs and the iPSC-CCs.

31. The method of claim 30, wherein:
the iPSC-ECs are induced pluripotent stem cell-derived vascular endothelial cells (iPSC-vECs);
the iPSC-CCs are induced pluripotent stem cell-derived cardiomyocytes (iPSC-CMs), induced pluripotent stem cell-derived fibroblast cells (iPSC-FCs), or induced pluripotent stem cell-derived smooth muscle cells (iPSC-SMCs), or the iPSC-CCs are induced pluripotent stem cell-derived cardiomyocytes (iPSC-CMs); and/or
the iPSC-ECs are human iPSC-ECs (hiPSC-ECs), and/or the iPSC-CCs are human iPSC-CCs (hiPSC-CCs).

32. (canceled)

33. (canceled)

34. (canceled)

35. The method of claim 31, wherein the harvesting iPSC-ECs and iPSC-CCs or iPSC-CMs comprises harvesting iPSC-ECs and iPSC-CCs or iPSC-CMs in a ratio of 1:9,
wherein the harvesting iPSC-ECs and iPSC-CCs or iPSC-CMs comprises harvesting about 2,000 or 2,000 iPSC-ECs and about 18,000 or 18,000 iPSC-CCs or iPSC-CMs per organoid,
wherein the harvested iPSC-ECs and iPSC-CCs or iPSC-CMs are resuspended in about 200 μL or 200 μL of the formulation in each well of the well plate such that cardiac organoids with about 20,000 or 20,000 cells are generated, and/or
wherein each generated organoid has about 2,000 or 2,000 iPSC-ECs and about 18,000 or 18,000 iPSC-CCs or iPSC-CMs.

36. (canceled)

37. (canceled)

38. (canceled)

39. The method of claim 31, wherein the harvesting iPSC-ECs and iPSC-CCs or iPSC-CMs comprises harvesting about 1,000 or 1,000 iPSC-ECs and about 9,000 or 9,000 iPSC-CCs or iPSC-CMs per organoid,
wherein the harvested iPSC-ECs and iPSC-CCs or iPSC-CMs are resuspended in about 200 μL or 200 μL of the formulation in each well of the well plate such that cardiac organoids with about 10,000 or 10,000 cells are generated, and/or
wherein each generated organoid has about 1,000 or 1,000 iPSC-ECs and about 9,000 or 9,000 iPSC-CCs or iPSC-CMs.

40. (canceled)

41. (canceled)

42. The method of claim 30, wherein the well plate is centrifuged for about 10 or 10 seconds at about 200× or 200× gravity to aggregate the organoids at the bottom of the wells,
wherein the aggregated organoids are cultured in the formulation at 37° C. under 5% CO₂ condition indefinitely, and/or
wherein the changing the formulation three times a week comprises conducting a 75% media change of the formulation to prevent aspiration of the organoids from wells.

43. (canceled)

44. (canceled)

45. A method of producing induced pluripotent stem cell (iPSC)-derived cardiac organoids containing iPSC-derived endothelial cells (iPSC-ECs), iPSC-derived cardiac fibroblasts (iPSC-CFs), and iPSC-derived cardiomyocytes (iPSC-CMs), the method comprising:
harvesting iPSC-ECs, iPSC-CFs, and iPSC-CMs from a well plate under a sterile cell culture environment;
resuspending the harvested iPSC-ECs, iPSC-CFs, and iPSC-CMs in the formulation of claim 1 to generate cardiac organoids;
centrifuging the well plate to aggregate the organoids at a bottom of each well of the well plate; and
culturing the organoids with the formulation, changing the formulation about three times a week,
wherein the formulation is optimized for growth of the iPSC-ECs, the iPSC-CFs, and the iPSC-CMs.

46. The method of claim 45, wherein the iPSC-ECs are induced pluripotent stem cell-derived vascular endothelial cells (iPSC-vECs), and/or wherein the iPSC-ECs are human iPSC-ECs (hiPSC-ECs), the iPSC-CFs are human iPSC-CFs (hiPSC-CFs), and the iPSC-CMs are human iPSC-CMs (hiPSC-CMs).

47. (canceled)

48. The method of claim 45, wherein the harvesting iPSC-ECs, the iPSC-CFs, and iPSC-CMs comprises harvesting iPSC-ECs, the iPSC-CFs, and iPSC-CMs in a ratio of 1:1:8.
wherein the harvesting iPSC-ECs, the iPSC-CFs, and iPSC-CMs comprises harvesting about 2,000 or 2,000 iPSC-ECs, harvesting about 2,000 or 2,000 iPSC-CFs, and about 16,000 or 16,000 iPSC-CMs per organoid,
wherein the harvested iPSC-ECs, iPSC-CFs, and iPSC-CMs are resuspended in about 200 μL or 200 μL of the formulation in each well of the well plate such that cardiac organoids with about 20,000 or 20,000 cells are generated, and/or
wherein each generated organoid has about 2,000 or 2,000 iPSC-ECs, about 2,000 or 2,000 iPSC-CFs, and about 16,000 or 16,000 iPSC-CMs

49. (canceled)

50. (canceled)

51. (canceled)

52. The method of claim 45, wherein the harvesting iPSC-ECs, iPSC-CFs, and iPSC-CMs comprises harvesting about 1,000 or 1,000 iPSC-ECs, about 1,000 or 1,000 iPSC-CFs and about 8,000 or 8,000 iPSC-CMs per organoid,
wherein the harvested iPSC-ECs, iPSC-CFs, and iPSC-CMs are resuspended in about 200 μL or 200 μL of the formulation in each well of the well plate such that cardiac organoids with about 10,000 or 10,000 cells are generated, and/or
wherein each generated organoid has about 1,000 or 1,000 iPSC-ECs, about 1,000 or 1,000 iPSC-CFs, and about 8,000 or 8,000 iPSC-CMs.

53. (canceled)

54. (canceled)

55. The method of claim 45, wherein the well plate is centrifuged for about 10 or 10 seconds at about 200× or 200× gravity to aggregate the organoids at the bottom of the wells,
wherein the aggregated organoids are cultured in the formulation at 37° C. under 5% CO₂ condition indefinitely, and/or
wherein the changing the formulation three times a week comprises conducting a 75% media change of the formulation to prevent aspiration of the organoids from wells

56. (canceled)

57. (canceled)
